(19) United States
(12) Patent Application Publication
(10) Pub. No.: US 2024/0361317 A1
(43) Pub. Date: Oct. 31, 2024

(54) PROBES BASED ON VOLATILE ORGANIC COMPOUNDS AND USES THEREOF FOR THE DIAGNOSIS AND PROGNOSIS OF PATHOLOGICAL CONDITIONS

(71) Applicants: Centre National de la Recherche Scientifique, PARIS (FR); Université de Poitiers, POITIERS CEDEX (FR); CENTRE HOSPITALIER UNIVERSITAIRE DE POITIERS, POITIERS (FR)

(72) Inventors: Pauline POINOT, POITIERS (FR); Sébastien PAPOT, POITIERS (FR); Justin LANGE, JAUNAY-MARIGNY (FR); Fabiola DJAGO, POITIERS (FR); Estelle BLOCHOUSE, POITIERS (FR)

(21) Appl. No.: 18/573,948

(22) PCT Filed: Jun. 22, 2022
(86) PCT No.: PCT/EP2022/066960

§ 371 Date: Dec. 22, 2023

(30) Foreign Application Priority Data
Jun. 22, 2021 (FR) 2106631
Mar. 17, 2022 (FR) 2202352

(51) Int. Cl. G01N 33/569; G01N 33/574

(52) U.S. Cl. CPC G01N 33/56983; G01N 33/57488; G01N 2333/924

(57) ABSTRACT

The present invention relates to a composition comprising: —either at least ethyl-β-D-glucuronide, the ethyl part of which comprises at least one non-radioactive isotope (or ethyl-β-D-glucuronide probe) and at least ethyl-β-D-galactopyranoside (or ethyl-β-D-galactopyranoside probe), the ethyl part of which comprises at least one non-radioactive isotope; —or at least ethyl-α-mannopyranoside, the ethyl part of which comprises at least one non-radioactive isotope (or ethyl-β-D-glucuronide probe); for use thereof as a diagnostic and/or prognostic agent.

The present invention relates to probes based on volatile organic compounds, compositions comprising these probes and their uses for the diagnosis and prognosis of pathological conditions. The present invention also relates to a method for diagnosing these pathological conditions by the use of said probes.

An early, precise, reliable diagnosis is one of the keys to ensuring the recovery of patients, regardless of their pathological condition. Until now, the diagnosis of a patient has resulted either from the clinical examination of the individual when the individual has particular symptoms, or from a participation in a national screening program. That patient then enters medical protocols involving an imaging analysis, which remain improvable in terms of 1) their low accessibility, 2) their specificity, 3) their high cost, and 4) their sensitivity.

“Volatolomics”, a discipline which studies the volatile organic compounds (VOCs) produced by biological systems, is an emerging scientific field which makes it possible to explore real-time metabolic processes. Many endogenous VOCs present in the breath, perspiration, urine, or stools of the individuals may be potential markers of various diseases, allowing both their diagnosis and the categorization of patients in the context of customized therapies. However, the clinical use of “volatolomics” remains limited because of high interindividual variability and interlaboratory disparity for the sampling and analysis of VOCs in fluids or biological gases.

Recently, biosensors for medical or biological purposes have been developed, these being based on detection by fluorescence, electrochemical or luminescence. However, these biosensors have the disadvantage of containing radioactive elements.

Therefore, there is a need for simple and sensitive means for diagnosis, based on probes derived from biological molecules and not containing any radioactive element. The aim of the present invention is to provide a composition comprising enzyme-sensitive probes, programmed to map the specific enzymes of one (more) pathological condition(s), to diagnose diseases early and with certainty, after administering the probes in the subjects to be diagnosed.

The present invention also aims to provide a composition for the diagnosis of subjects, making it possible to track the subjects very regularly in the course of their therapy and thus to assist the medical personnel in their decision-making as to the choice of the most effective protocol.

Another object of the present invention is to provide a simple, inexpensive, very sensitive, painless diagnostic method accessible to a large number, in order to be able to improve the treatment of malign pathological conditions.

The aim of the present invention is to provide a composition for diagnosis, making it possible to considerably reduce the inter-individual variability encountered when tracking endogenous markers.

Thus, the present invention relates to a composition comprising:
at least ethyl-β-D-glucuronide, the ethyl part of which comprises at least one non-radioactive isotope (or ethyl-β-D-glucuronide probe);
at least ethyl-N-acetylglucosamide, the ethyl part of which comprises at least one non-radioactive isotope (or ethyl-N-acetylglucosamide probe); and
at least ethyl-β-D-galactopyranoside (or ethyl-β-D-galactopyranoside probe), the ethyl part of which comprises at least one non-radioactive isotope;
   for use thereof as a diagnostic and/or prognostic agent.

Thus, the present invention relates to a composition comprising:
either at least ethyl-β-D-glucuronide, the ethyl part of which comprises at least one non-radioactive isotope (or ethyl-β-D-glucuronide probe) and at least ethyl-β-D-galactopyranoside (or ethyl-β-D-galactopyranoside probe), the ethyl part of which comprises at least one non-radioactive isotope;
either at least ethyl-α-mannopyranoside, the ethyl part of which comprises at least one non-radioactive isotope (or ethyl-α-mannopyranoside probe); for its use as diagnostic and/or prognostic agent.

The present invention also relates to a composition (C1) comprising:
at least ethyl-β-D-glucuronide, the ethyl part of which comprises at least one non-radioactive isotope (or ethyl-β-D-glucuronide probe) and
at least ethyl-β-D-galactopyranoside (or ethyl-β-D-galactopyranoside probe), the ethyl part of which comprises at least one non-radioactive isotope, for use thereof as a diagnostic and/or prognostic agent.

The present invention also relates to a composition (C2) comprising at least ethyl-α-mannopyranoside, the ethyl part of which comprises at least one non-radioactive isotope (or ethyl-α-mannopyranoside probe), for use thereof as a diagnostic and/or prognostic agent.

### Composition or Cocktail According to the Invention

The aforementioned composition therefore comprises several compounds or probes, intended for use as an agent for the diagnosis or prognosis of a pathological condition.

This composition is also designated subsequently by the term “cocktail” or “probe cocktail”. This composition is therefore used to diagnose or predict a pathological condition, as explained below. As compositions according to the invention, mention may therefore be made, for example, of the abovementioned compositions C1 or C2.

As mentioned above, the composition used according to the invention comprises for example at least two probes, in particular at least three probes, namely ethyl-β-D-glucuronide probes, ethyl-N-acetylglucosamide and ethyl-β-D-galactopyranoside, each of these probes (or compounds) comprising at least one non-radioactive isotope in the ethyl part.

As mentioned above, a composition C1, used according to the invention, also called “cocktail 1”, comprises at least two probes, namely ethyl-β-D-glucuronide and ethyl-β-D-galactopyranoside probes, each of these probes (or compounds) comprising at least one non-radioactive isotope in the ethyl part. According to one embodiment, this composition comprises for example the ethyl-N-acetylglucosamide probe.

Another composition used according to the invention, namely composition C2, also called “cocktail 2”, comprises at least the ethyl-α-mannopyranoside probe, preferably in combination with at least one other probe such as for example the ethyl-α-D-glucopyranoside probe or the ethyl-1-α-L-fucopyranoside probe.

Ethyl-β-D-glucuronide corresponds to the following formula:

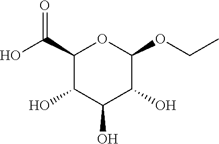

and targets the β-glucuronidase enzyme.

Ethyl-N-acetylglucosamide corresponds to the following formula:

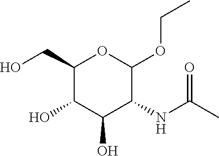 [SMILES: CCOC1O[C@H](CO)[C@@H](O)[C@H](O)[C@H]1NC(C)=O]

and targets the enzyme N-acetylglucosaminidase.

Ethyl-β-D-galactopyranoside corresponds to the following formula:

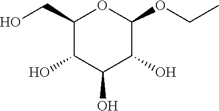

and targets the β-galactosidase enzyme.

As explained above, the probes of the composition according to the invention comprise, in their ethyl part, at least one non-radioactive isotope. As non-radioactive isotopes, mention may be made, for example, of deuterium (²D), carbon-13 (¹³C) or oxygen 18 (¹⁸O). These probes are therefore compounds derived from the compounds corresponding to the abovementioned formulae, in which at least one hydrogen and/or carbon and/or oxygen atom of their ethyl parts are replaced by an isotope. The ethyl part of the probes used according to the invention is therefore marked with one or more non-radioactive isotopes on one or more positions (²D, ¹³C or ¹⁸O).

According to one embodiment, ethyl-β-D-glucuronide, the ethyl part of which comprises at least one non-radioactive isotope present in the composition according to the invention, is the probe D₅-ethyl-β-D-glucuronide, corresponding to the following formula (1):

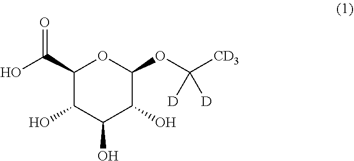 [SMILES: [2H]C([2H])([2H])C([2H])([2H])O[C@@H]1O[C@H](C(=O)O)[C@@H](O)[C@H](O)[C@H]1O]

this probe targeting the enzyme β-glucuronidase.

According to one embodiment, ethyl-N-acetylglucosamide, the ethyl part of which comprises at least one non-radioactive isotope present in the composition according to the invention is the probe¹³C-D₅-ethyl-N-acetylglucosamide, corresponding to the following formula (2):

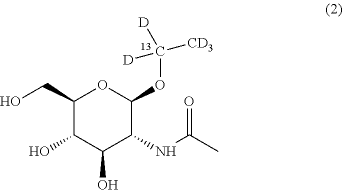 [SMILES: [2H]C([2H])([2H])C([2H])([2H])O[C@@H]1O[C@H](CO)[C@@H](O)[C@H](O)[C@H]1NC(C)=O]

this probe targeting the enzyme N-acetyl-glucosaminidase.

According to one embodiment, ethyl-β-D-galactopyranoside, the ethyl part of which comprises at least one non-radioactive isotope present in the composition according to the invention, is the probe D₂-ethyl-β-D-galactopyranoside, corresponding to the following formula (3):

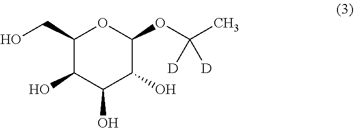 [SMILES: [2H]C([2H])(C)O[C@@H]1O[C@H](CO)[C@H](O)[C@H](O)[C@H]1O]

this probe targeting the enzyme β-galactosidase.

Ethyl-1-α-L-fucopyranoside corresponds to the following formula:

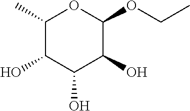

and targets the enzyme α-L-fucosidase.

Ethyl-N-glucopyranoside corresponds to the following formula:

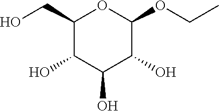

and targets the enzyme α- or β-glucosidase.

Ethyl-mannopyranoside corresponds to the following formula:

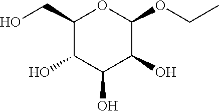

and targets the enzyme α- or β-mannosidase.

Ethyl-α-D-glucopyranoside corresponds to the following formula:

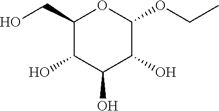

and targets the enzyme α-glucosidase.

Ethyl-α-mannopyranoside corresponds to the following formula:

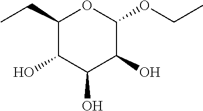

and targets the enzyme α-mannosidase.

According to one embodiment, ethyl-1-α-L-fucopyranoside, the ethyl part of which comprises at least one non-radioactive isotope present in the composition according to the invention, is the probe D₄-ethyl-1-α-L-fucopyranoside, corresponding to the following formula (4):

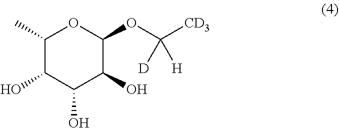 [SMILES: [2H]C(O[C@@H]1O[C@@H](C)[C@@H](O)[C@@H](O)[C@@H]1O)C([2H])([2H])[2H]]

this probe targeting the enzyme α-L-fucosidase.

According to one embodiment, ethyl-glucopyranoside, the ethyl part of which comprises at least one non-radioactive isotope present in the composition according to the invention, is the probe D₄-ethyl-glucopyranoside, corresponding to the following formula (5):

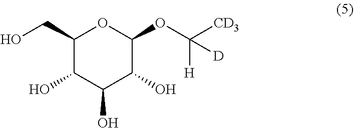 [SMILES: [2H]C(O[C@@H]1O[C@H](CO)[C@@H](O)[C@H](O)[C@H]1O)C([2H])([2H])[2H]]

According to one embodiment, ethyl-mannopyranoside, the ethyl part of which comprises at least one non-radioactive isotope present in the composition according to the invention, is the probe D₅-ethyl-mannopyranoside, corresponding to the following formula (6):

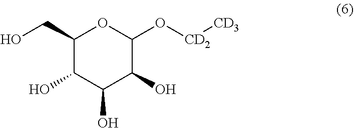

All the probes can be randomly synthesized with the ethanol isotopes that: are ethanol-D2, ethanol-D3, ethanol-D4, ethanol-D5, ethanol-13C-D5, ethanol-13C2-D5, ethanol-13C or ethanol-13C2.

Thus, the present invention, departing from the conventional research of endogenous VOCs, is based on the ex vivo use of multi-modal probes, including in their structure a VOC labeled with one or more stable isotope(s). These interacting probes are selectively hydrolyzed in injured tissues via the activity of enzymes specific for these media. The VOCs thus released in turn become exogenous markers characteristic of the metabolism of the injured tissue and are detected ex vivo in a biological sample according to a established and detailed protocol, as explained below.

A first proof of concept targeting β-glucuronidase, a tumor enzyme marker, showed the relevance of the use of a deuterium-labeled ethanol probe for detecting various types of solid tumors in vivo and monitoring their evolution during chemotherapy. However, in a context of clinical research, the targeting of a single enzyme may lead to a significant number of false positives and false negatives.

The present invention is therefore based on the use of a cocktail of interacting probes, each targeting an enzymatic marker specific for one or more pathological conditions, in order to reduce the risk of erroneous diagnosis and to predict the efficacy of associated therapies. Depending on the probes activated, this strategy provides information on the nature of the pathological condition (e.g. solid tumor, inflammatory syndrome, bacterial infection, viral infection), its location, its aggressiveness, its development stage, and its response to treatments.

In the context of the present invention, probes with VOCs targeting glycosidase enzymes are therefore used. These enzymes are expressed in the injured tissues (e.g. tumor or infectious micro-environment), but are not, or very little, expressed/secreted by healthy cells.

After hydrolysis, these probes release a labeled ethanol molecule.

Furthermore, the VOC-based probes according to the invention offer a heretofore unmatched sensitivity. As an example, the first results obtained with the probe targeting β-glucuronidase show that this enzyme can be detected in plasma at concentrations of less than 5.10⁻¹³ M. A fluorescent biosensor developed recently had a LOD of 5.10⁻⁹ M for this same enzyme.

According to one embodiment, the aforementioned composition, in particular composition C1 or “cocktail 1”, further comprises at least one other labeled compound selected from the group consisting of: ethyl-1-α-L-fucopyranoside, the ethyl part of which comprises at least one non-radioactive isotope, ethyl-β-D-glucopyranoside, the ethyl part of which comprises at least one non-radioactive isotope, ethyl-mannopyranoside, the ethyl part of which comprises at least one non-radioactive isotope, and mixtures thereof.

According to one embodiment, the aforementioned composition, in particular composition C1 or “cocktail 1”, further comprises ethyl-N-acetylglucosamide.

According to one embodiment, the composition of the invention comprises at least the aforementioned probes (1), (2) and (3), in combination with at least one of the probes (4), (5) and/or (6) as defined above.

According to one embodiment, the composition of the invention C1 comprises at least the aforementioned probes (1) and (3), in combination with at least one of the probes (2), (4), (5) and/or (6) as defined above.

According to one embodiment, the composition of the invention C2 comprises at least the aforementioned probe (6), in combination with at least one of the probes (4) and/or (5) as defined above.

According to one embodiment, composition C1 comprises at least ethyl-β-D-glucuronide, the ethyl part of which comprises at least one non-radioactive isotope, ethyl-N-acetylglucosamide, the ethyl part of which comprises at least one non-radioactive isotope, ethyl-β-D-galactopyranoside, the ethyl part of which comprises at least one non-radioactive isotope, and ethyl-1-α-L-fucopyranoside, the ethyl part of which comprises at least one non-radioactive isotope.

This cocktail according to the invention C1-1 thus preferably comprises ethyl-β-d-glucuronide targeting β-glucuronidase, ethyl-N-acetylglucosamide targeting N-acetyl glucosaminidase, ethyl-β-D-galactopyranoside targeting β-galactosidase and ethyl-1-α-L-fucopyranoside targeting α-L-fucosidase, each of these probes being modified as explained above, by the presence of at least one non-radioactive isotope in their ethyl parts.

According to one embodiment, composition C1 comprises at least ethyl-β-D-glucuronide, the ethyl part of which comprises at least one non-radioactive isotope, ethyl-β-D-galactopyranoside, the ethyl part of which comprises at least one non-radioactive isotope and ethyl-α-D-glucopyranoside, the ethyl part of which comprises at least one non-radioactive isotope

This cocktail according to the invention C1-2 thus preferably comprises ethyl-β-d-glucuronide targeting β-glucuronidase, ethyl-β-D-galactopyranoside targeting β-galactosidase and ethyl-α-D-glucopyranoside targeting α-glucosidase, each of these probes being modified as explained above, by the presence of at least one non-radioactive isotope in their ethyl parts.

According to one embodiment, composition C2 comprises at least ethyl-α-D-glucopyranoside, the ethyl part of which comprises at least one non-radioactive isotope and ethyl-α-mannopyranoside, the ethyl part of which comprises at least one non-radioactive isotope.

This cocktail according to the invention C2-1 thus preferably comprises ethyl-α-D-glucopyranoside targeting α-glucosidase and ethyl-α-mannopyranoside targeting α-mannosidase, each of these probes being modified as explained above, by the presence of at least one non-radioactive isotope in their ethyl parts.

According to one embodiment, composition C2 comprises at least ethyl-1-α-L-fucopyranoside, the ethyl part of which comprises at least one non-radioactive isotope and ethyl-α-mannopyranoside, the ethyl part of which comprises at least one non-radioactive isotope.

This cocktail according to the invention C 2-2 thus preferably comprises ethyl-1-α-L-fucopyranoside targeting α-L-fucosidase and ethyl-α-mannopyranoside targeting α-mannosidase, each of these probes being modified as explained above, by the presence of at least one non-radioactive isotope in their ethyl parts.

According to one embodiment, the composition comprises at least ethyl-β-D-glucuronide, the ethyl part of which comprises at least one non-radioactive isotope, ethyl-N-acetylglucosamide, the ethyl part of which comprises at least one non-radioactive isotope, ethyl-β-D-galactopyranoside, the ethyl part of which comprises at least one non-radioactive isotope, ethyl-1-α-L-fucopyranoside, the ethyl part of which comprises at least one non-radioactive isotope, ethyl-glucopyranoside, the ethyl part of which comprises at least one non-radioactive isotope and ethyl-mannopyranoside, the ethyl part of which comprises at least one non-radioactive isotope.

The cocktail according to the invention thus preferably comprises ethyl-β-d-glucuronide targeting β-glucuronidase, ethyl-N-acetylglucosamide targeting N-acetyl glucosaminidase, ethyl-β-D-galactopyranoside targeting β-galactosidase, ethyl-1-α-L-fucopyranoside targeting α-L-fucosidase, ethyl-glucopyranoside targeting α-glucosidase or β-glucosidase and ethyl-mannopyranoside targeting α-mannosidase or β-mannosidase, each of these probes being modified as explained above, by the presence of at least one non-radioactive isotope in their ethyl parts.

According to one embodiment, the composition according to the invention comprises the abovementioned probes corresponding to formulae (1), (2), (3), (4), (5) and (6).

### Use of the Composition or Cocktail According to the Invention

The present invention also relates to the use of the composition as defined above, namely a composition comprising:
at least ethyl-β-D-glucuronide, the ethyl part of which comprises at least one non-radioactive isotope;
at least ethyl-N-acetylglucosamide, the ethyl part of which comprises at least one non-radioactive isotope; and
at least ethyl-β-D-galactopyranoside, the ethyl part of which comprises at least one non-radioactive isotope;
   as an agent for the diagnosis and/or prognosis ex vivo of a pathological condition selected from the group consisting of pathological conditions involving an inflammatory reaction in response to a lesion or infection of the organism, chronic inflammatory diseases and acute infections.

The present invention also relates to the use of the composition as defined above, namely a composition comprising:
either at least ethyl-β-D-glucuronide, the ethyl part of which comprises at least one non-radioactive isotope (or ethyl-β-D-glucuronide probe) and at least ethyl-β-D-galactopyranoside (or ethyl-β-D-galactopyranoside probe), the ethyl part of which comprises at least one non-radioactive isotope;
either at least ethyl-α-mannopyranoside, the ethyl part of which comprises at least one non-radioactive isotope (or ethyl-α-mannopyranoside); as an agent for the diagnosis and/or prognosis ex vivo of a pathological condition selected from the group consisting of pathological conditions involving an inflammatory reaction in response to a lesion or infection of the organism, chronic inflammatory diseases and acute infections.

The present invention also relates to the use of the composition as defined above, namely a composition C1 or C2, as an agent for the diagnosis and/or prognosis ex vivo of a pathological condition selected from the group consisting of pathological conditions involving an inflammatory reaction in response to a lesion or infection of the organism, chronic inflammatory diseases and acute infections.

As explained below, the compositions according to the invention, and as defined above, in particular compositions C1 or C2, and comprising the abovementioned probes, are used for the diagnosis and/or prognosis ex vivo of subjects (human or animal). These compositions can be intended in particular for the categorization of subjects or patients, according to the progress, severity, and evolution of the pathological condition, but also to diagnose, possibly early, a pathological condition, for example in the context of a screening campaign of a given population of subjects or patients.

According to one embodiment, the composition used as an agent for the diagnosis and/or prognosis ex vivo of the abovementioned pathological condition is as defined above, and comprises in particular the probes (1), (2) and (3).

According to one embodiment, the composition used an agent for the diagnosis and/or prognosis ex vivo of the abovementioned pathological condition is composition C1 or C2. According to one embodiment, the composition used an agent for the diagnosis and/or prognosis ex vivo of the abovementioned pathological condition comprises the aforementioned probes (1), (2), (3), (4), (5) and (6).

### Pathological Conditions

As indicated above, the pathological conditions that can be diagnosed and/or predicted by the cocktail of probes according to the invention are pathological conditions involving an inflammatory reaction in response to a lesion or infection of the organism, chronic inflammatory diseases, and acute infections. Among these pathological conditions, mention may in particular be made of inflammatory diseases, bacterial infections or even autoimmune diseases.

The term “pathological conditions involving an inflammatory reaction in response to a lesion or infection of the organism” denotes in particular cancers, pulmonary ARDS and fibrosis, or Parkinson's disease.

The term “chronic inflammatory diseases” denotes in particular Crohn's disease, eczema, as well as autoimmune diseases such as type 1 diabetes, vitiligo, multiple sclerosis, or rheumatoid arthritis.

The term “acute infections” in particular refers to viral infections (respiratory syncytial, SARS-COV, HIV, herpes simplex virus and all

emerging viruses) and bacterial infections (Escherichia Coli, Staphylococcus, Pseudomonas, Propionibacterium acnes).

Inflammatory disease, also called inflammatory illness, can be any disease resulting from an abnormal reaction of the immune system.

The inflammatory disease may be an auto-inflammatory disease, an autoimmune disease or an inflammatory condition of indeterminate origin.

Inflammatory illnesses are defined by tissue attack due to an immune system dysfunction. Thus, among them, mention may be made of atherosclerosis, rheumatoid arthritis, pulmonary inflammation, chronic inflammatory bowel disease, sepsis, severe sepsis, septic shock or cancer.

The inflammatory disease is thus preferably selected from the group consisting of atherosclerosis, rheumatoid arthritis, pulmonary inflammation, chronic inflammatory bowel disease, sepsis, severe sepsis, septic shock, cancer and combinations thereof.

Atherosclerosis (also called arteriosclerosis) is a disease affecting the arteries and characterized by the appearance of atheromatous plaques on the inner wall of the arteries.

Rheumatoid arthritis (or RA) is a systemic disease of the connective tissue characterized by chronic articular inflammation that sporadically appears.

Pulmonary inflammation in particular comprises inflammatory pulmonary disease and/or pulmonary allergy.

Inflammatory pulmonary disease in particular comprises acute lung injury (ALI, also called “moderate ARDS”), acute respiratory distress syndrome (or ARDS), and transfusion related acute lung injury (or TRALI).

Pulmonary allergy, also called respiratory allergy, is a disease alternating an acute phase (also called asthma) and a chronic phase.

Chronic inflammatory bowel disease (or IBD) can be selected from Crohn's disease and ulcerative colitis.

An excessive inflammatory reaction, in response to an infection, causes sepsis and its most serious forms, severe sepsis and septic shock.

The cancer cell will cause inflammation and invade the neighboring cells to create a climate conducive to the growth of nearby cells. The cancer tumor therefore grows little by little. Specifically, the cancer cell uses inflammation to progress.

According to one embodiment, the aforementioned pathological condition is selected from the group consisting of cancers, bacterial and viral infections, nosocomial infections, autoimmune diseases, respiratory inflammations, chronic inflammations, neurodegenerative diseases, and chronic age-related disorders.

According to the invention, among cancers, mention may be made, for example, of solid cancer tumors, such as for example lung cancers (lungs, bronchi and naso-pharynx), gynecological (breast, ovarian, uterus) and gastrointestinal (liver, intestine, colon, pancreas).

Preferably, the cancer is selected from the group consisting of breast cancer, lung cancer, pancreatic cancer, ovarian cancer, uterine cancer, nasopharyngeal cancer, intestine cancer, colon cancer, bladder cancer and brain cancer.

As bacterial and viral infections, mention may be made of infections due, for example, to Streptococcus pneumoniae, Escherichia coli, Staphylococcus aureus, Klebsiella pneumoniae, Acinetobacter baumannii and Pseudomonas aeruginosa, Salmonella, Listeria monocytogenes, Chlamydia, Treponema pallidum, SARS-COV, HIV, syncytial virus, herpes simplex virus and all emerging viruses.

As nosocomial infections, mention may be made, for example, of infections due to Staphylococcus aureus, Escherichia coli and Propionibacterium acnes.

As auto-immune diseases, mention may be made, for example, of type 1 diabetes, vitiligo, multiple sclerosis or rheumatoid arthritis.

As respiratory inflammations, mention may be made, for example, of pulmonary fibrosis, and in particular acute hypoxemic respiratory failure or acute respiratory distress syndrome (ARDS).

Mention may be made, as chronic inflammations, for example, of Crohn's disease.

As neurodegenerative diseases, mention may be made, for example, of Alzheimer's disease or Parkinson's disease.

As chronic age-related disorders, mention may be made, for example, of type 2 diabetes, osteoarthritis and osteoporosis.

The present invention also relates to the aforementioned composition, in particular comprising the probes (1), (2) and (3), or the composition C1 or C2, for its use as an agent for diagnosis and/or prognosis of cancers.

The present invention also relates to a composition as defined above comprising the probes (1), (2), (3) and (4), for its use as an agent for diagnosis and/or prognosis of cancers.

The present invention also relates to a composition as defined above comprising the probes (1), (2), (3), (4) and (6), for its use as an agent for diagnosis and/or prognosis of cancers.

The present invention also relates to a composition C1 or C2 as defined above, for its use as an agent for diagnosis and/or prognosis of cancers.

The present invention also relates to a composition as defined above comprising the probes (1), (2), (3) and (5), for its use as an agent for diagnosis and/or prognosis of viral and bacterial infections and chronic inflammatory diseases such as osteoarthritis or diabetes.

The present invention also relates to a composition C1 or C2 as defined above, for its use as an agent for diagnosis and/or prognosis of viral and bacterial infections and chronic inflammatory diseases such as osteoarthritis or diabetes.

### Method for Diagnosing a Pathological Condition

The present invention also relates to a method for diagnosing a pathological condition in a subject, said pathological condition being selected from the group consisting of pathological conditions involving an inflammatory reaction in response to a lesion or infection of the organism, chronic inflammatory diseases, and acute infections, said method comprising the following steps:
a) adding, in a biological sample of said subject, a composition comprising:
   at least ethyl-β-D-glucuronide, the ethyl part of which comprises at least one non-radioactive isotope;
   at least ethyl-N-acetylglucosamide, the ethyl part of which comprises at least one non-radioactive isotope; and
   at least ethyl-β-D-galactopyranoside, the ethyl part of which comprises at least one non-radioactive isotope;
b) measuring the amount of labeled ethanol, released in the gas phase, c) comparing the values obtained in step b) with a corresponding standard control value, and
d) deducing therefrom whether the subject is affected by said pathological condition.

The aforementioned diagnostic method is preferably an ex vivo method.

The present invention also relates to a method for diagnosing a pathological condition in a subject, said pathological condition being selected from the group consisting of pathological conditions involving an inflammatory reaction in response to a lesion or infection of the organism, chronic inflammatory diseases, and acute infections, said method comprising the following steps:
a) adding, in a biological sample of said subject, a composition comprising:
   either at least ethyl-β-D-glucuronide, the ethyl part of which comprises at least one non-radioactive isotope (or ethyl-β-D-glucuronide probe) and at least ethyl-β-D-galactopyranoside (or ethyl-β-D-galactopyranoside probe), the ethyl part of which comprises at least one non-radioactive isotope;
   or at least ethyl-α-mannopyranoside, the ethyl part of which comprises at least one non-radioactive isotope (or ethyl-α-mannopyranoside probe);
b) measuring the amount of labeled ethanol, released in the gas phase, c) comparing the values obtained in step b) with a corresponding standard control value, and
d) deducing therefrom whether the subject is affected by said pathological condition.

The present invention also relates to a method for diagnosing a pathological condition in a subject, said pathological condition being selected from the group consisting of pathological conditions involving an inflammatory reaction in response to a lesion or infection of the organism, chronic inflammatory diseases, and acute infections, said method comprising the following steps:
a) adding, in a biological sample of said subject, a composition C1 or C2 as defined above,
b) measuring the amount of labeled ethanol, released in the gas phase, c) comparing the values obtained in step b) with a corresponding standard control value, and
d) deducing therefrom whether the subject is affected by said pathological condition.

According to one embodiment, steps a) to d) can be carried out for two identical samples of the same subject in parallel, with one side being a composition C1 and the other a composition C2.

According to one embodiment, the aforementioned diagnostic method comprises the implementation of the aforementioned composition, in particular comprising the probes (1), (2) and (3), for the diagnosis of cancers.

According to one embodiment, the aforementioned diagnostic method comprises the implementation of the aforementioned composition comprising the probes (1), (2), (3), and (4), for the diagnosis of cancers.

According to one embodiment, the aforementioned diagnostic method comprises the implementation of the aforementioned composition comprising the probes (1), (2), (3), (4), and (6), for the diagnosis of cancers.

According to one embodiment, the aforementioned diagnostic method comprises the implementation of the aforementioned composition C1 or C2, for the diagnosis of cancers.

According to one embodiment, the aforementioned diagnostic method comprises the implementation of the aforementioned composition comprising the probes (1), (2), (3) and (5), for the diagnosis of viral and bacterial infections and chronic inflammatory diseases such as osteoarthritis or diabetes.

According to one embodiment, the aforementioned diagnostic method comprises the implementation of the aforementioned composition C1 or C2, for the diagnosis of viral and bacterial infections and chronic inflammatory diseases such as osteoarthritis or diabetes.

### Subject

According to one embodiment, the subject is a human or an animal.

The subject is preferably a mammal, for example a human subject or a non-human mammal, such as a cat, dog, monkey, rabbit, mouse or rat.

The human subject may be a male or a female of any age, such as an infant, a child, an adolescent, an adult or an elderly person.

The subject is preferably a subject suffering or at risk of suffering from a pathological condition as defined above, in particular an inflammatory disease.

A subject at risk of suffering an inflammatory disease is for example a subject with a family history of inflammatory disease, having already suffered at least one inflammatory disease, having genetic risk factors, metabolic and/or lifestyle factors and/or having at least one symptom that can be a precursor to an inflammatory disease.

### Biological Sample

The biological sample is preferably obtained from a subject as defined above. The biological sample is preferably selected from the group consisting of plasma, blood, tissue, saliva, urine, cerebrospinal fluid samples, and biopsies of said subject.

Step a) consists of placing a biological sample obtained from a subject in contact with the composition according to the invention, in particular the cocktail of probes as defined above. This putting in contact is carried out by adding the composition of the invention (or cocktail of probes) into said biological sample, by any means well known to the person skilled in the art.

For example, the composition is added to a container containing said sample, for example a plasma or blood sample.

The addition of the composition of the invention into a biological sample can be carried out directly in the biological sample or after treatment of the biological sample.

According to one embodiment, the biological sample, preferably a volume of 1 mL for the fluids and several hundred grams for the tissues, is maintained, after the addition of said composition, at −20° C. until the analysis corresponding to the measurement step b) of the aforementioned method. It should not be unfrozen before analysis.

According to one embodiment, the biological sample obtained from the subject is also added to a buffer solution such as, for example, an acetate buffer (in particular pH 5.5, 0.1 mol/L, containing a mixture of sodium acetate and acetic acid), used to detect the marker enzymes of solid tumors in biological samples.

According to one embodiment, the biological sample obtained from the subject is also added to a protease inhibitor (e.g., Complete, EDTA-free protease inhibitor cocktail, Roche). By way of example, to detect solid tumor marker enzymes in biological samples, the protease inhibitor, prepared with UP water at a concentration of 7×, is used at a final concentration of 1×.

Thus, this step a) may be preceded by a step of treating the biological sample.

“Treating the biological sample” here means at least one treatment step, for example selected from the group consisting of centrifugation, freezing and thawing cycle, and suspending in a buffer.

According to one embodiment, the composition of the invention comprises is prepared by adding the abovementioned probes in a buffer solution, in particular as defined above, preferably at a concentration of between 10⁻³ mol/L and 10⁻⁷ mol/L. Preferably, the probes are added with a volume of between 10 μL and 200 μL to obtain a total sample volume of between 25 μL and 300 μL (total volume including 10 μL to 250 μL of buffer, 10 μL to 250 μL of biological fluid or 5 mg to 5000 mg of tissue, and 1 μL to 35 μL of 1× protease inhibitor).

According to one embodiment, the sample in its entirety (that is including the biological sample, the probes and optionally the buffer solution and the protease inhibitor) is inserted into a vial of hermetically sealed inert material, such as for example a 2 μL vial with hermetic plug with insert.

For example, for the diagnosis of tumors from plasma samples, the probes (1-4) are prepared beforehand in the aforementioned buffer at a concentration of 5-10⁵ mol/L, and added with a volume of 10 μL to obtain a total sample volume of 100 μL (total volume including 62 μL of buffer, 25 μL of biological fluid and 3 μL of protease inhibitor). The sample in its entirety is for example inserted into a 2 μL vial with hermetic plug with insert. Step b) consists of measuring the amount of labeled ethanol, released in the gas phase. The labeled ethanol is ethanol released from the abovementioned probes and thus containing the labeled ethyl part as defined above.

According to one embodiment, this measuring step is carried out on at least three kinetic points comprised between 0 and 8 h after step a) corresponding to the addition of the composition of the invention. A kinetic point corresponds to a measurement carried out at an instant t for the same sample prepared under identical conditions. To obtain three kinetic points, three identical biological samples are prepared as explained above, into which the cocktail of probes according to the invention as defined above is added under identical conditions, and the measurement step b) according to the invention is then carried out at three distinct instants, t1, t2 and t3 spaced apart over time.

Thus, for example, a measurement is carried out 2 h after step a), another measurement is carried out 4 h after step a) and a last measurement is carried out 7 h after step a). These kinetic points are obtained by carrying out the measurements in 3 separate flasks containing the same biological sample and the same composition according to the invention. These kinetic points, produced at least in duplicate, then make it possible to obtain an enzymatic curve useful for the conclusion step concerning the diagnosis, as explained below.

After the probes have been placed in the presence of the ethyl part of which comprises at least one non-radioactive isotope with the biological sample, if this biological sample contains at least one of the enzymes targeted by said probes, the probes will then be cut by the enzymes by hydrolysis, which will release the —O-Et part from said probes as explained above and therefore will result in the release of ethanol gas comprising at least one non-radioactive isotope.

For example, the aforementioned probe (1) allows the release of labeled ethanol in the form CD3-CD2-OH, the aforementioned probe (2) allows the release of labeled ethanol in the form CD₃-¹³CD₂-OH, the aforementioned probe (3) allows the release of labeled ethanol in the form CH3-CD2-OH, the aforementioned probe (4) allows the release of labeled ethanol in the form CD3-CHD-OH, the aforementioned probe (5) allows the release of labeled ethanol in the form CD3-CD2-OH, and the aforementioned probe (6) allows the release of labeled ethanol in the form ¹³CD3-¹³CD2-OH.

As explained above, the probes of the composition of the invention are selectively hydrolyzed in injured tissues via the activity of enzymes specific for these media. The labeled ethanol thus released becomes an exogenous marker characteristic of the metabolism of the injured tissue and is detected ex vivo in the biological sample. If the biological sample is that of a healthy subject, this sample not containing the targeted enzymes, the probes of the composition will therefore not be hydrolyzed and therefore no release of labeled ethanol will be obtained and therefore cannot be measured.

According to one embodiment, the biological sample is in a container such as a vial or flask, and, in the context of step a), the composition is added to said container, then said container is hermetically sealed.

According to one embodiment, the container is stirred for example at a vortex before the implementation of step b) of measuring the amount of ethanol released in the gas phase.

The measurement of the amount of ethanol released in the gas phase during step b) can be carried out by any measurement method or method well known to the person skilled in the art to measure the amount or concentration of ethanol marked in a gas phase such as, for example, solid-phase microextraction (SPME). According to one embodiment, step b) comprises a gas chromatography (GC) measurement coupled to a triple quadrupole equipped with an El source (GC-TQMS), or gas chromatography coupled to a high resolution mass spectrometer (GC-QTOFMS), or an artificial nose.

After step b) of measuring the amount of marked ethanol released, the values thus obtained are compared with a corresponding standard control value.

In step c), the results obtained in step b) are compared with a corresponding standard control value.

The results obtained in step b) are the amount, concentration, and/or proportion of labeled ethanol released in the gas phase, after adding the composition of the invention into the biological sample.

The term “corresponding” in the expression “corresponding standard control value” means that the amount, concentration, and/or proportion measured in the biological sample for the labeled ethanol is compared to a standard control value of the amount, concentration, and/or proportion of ethanol.

If it is the amount of ethanol that is measured in the biological sample, the standard control value is an amount.

If it is the concentration of ethanol that is measured in the biological sample, the standard control value is a concentration.

If it is the proportion of ethanol that is measured in the biological sample, the standard control value is a proportion.

A standard control value, also called reference value, may for example be the average, the maximum threshold value and/or the minimum threshold value of the amount, concentration, or proportion of ethanol measured in a sample from a healthy subject (after implementing steps a) and b) mentioned above), or the average, the maximum threshold value and/or the minimum threshold value of the amount, concentration, or proportion of ethanol measured in a blank sample, that is to say in a sample wherein the composition of the invention was added but not containing the target enzymes.

The values measured in the samples of healthy subjects or in blank samples are carried out using a measurement method similar to that used for the biological sample.

Two measurement methods are similar if, implemented on the same sample, they give an identical or similar result. In this respect, the calculation of the coefficient of variation (CV) is an important measure of the reliability of the method, which is acceptable if CV <20%.

The minimum threshold value and the maximum threshold value define a reference interval. The reference interval is generally defined so as to comprise 95% of the values obtained in the reference population.

Healthy subjects or individuals mean subjects or individuals with a good general health condition, in particular not suffering from pathological conditions as defined above.

The standard control value of ethanol can be the average, the maximum threshold value, and/or the minimum threshold value of the amount, concentration, or proportion of ethanol measured in the samples from healthy individuals, the samples having been added to the probe composition or cocktail according to the invention.

A standard control value may for example correspond to the average of the values obtained in the samples from 10 healthy subjects.

A subject will then be considered to be healthy when the ethanol value measured as explained above will be included in the standard deviation relative to the average of the values for the healthy subjects over all of the kinetic points as explained above.

In step d), it is deduced whether the subject is or is not afflicted with the aforementioned pathological condition.

In particular, if the quantity, concentration, and/or proportion of the measured quantity of labeled ethanol measured is greater than a corresponding standard control value (in particular than the maximum threshold value or than a set value), it is deduced therefrom that the presence of at least one enzyme targeted by the probes defined above, and therefore that the subject is afflicted with a pathological condition as defined above.

The standard deviation of the values for the diseased subjects should not be superimposed with the standard deviation of the values for the healthy subjects on at least one of the kinetic points (as explained above) in order to consider the targeted enzyme a marker of the pathological condition.

A subject is then considered to be sick when its measured ethanol value is included in the standard deviation relative to the average of the measured values for the diseased subjects on at least one of the kinetic points. This value should be at least 1.3 times greater than the average of the values measured for healthy subjects on at least one of the kinetic points.

According to one embodiment, when at the end of step d) it is concluded that the subject is afflicted by the aforementioned pathological condition, an additional step can be implemented consisting of a step of treating said subject, for example by administering to said subject a pharmaceutically acceptable amount of a compound or a composition intended for treating said pathological condition.

The present invention also relates to a method for diagnosing a pathological condition in a subject, said pathological condition being selected from the group consisting of pathological conditions involving an inflammatory reaction in response to a lesion or infection of the organism, chronic inflammatory diseases, and acute infections, said method comprising the following steps:
a) adding, in a blood sample of said subject, a composition comprising:
   either at least ethyl-β-D-glucuronide, the ethyl part of which comprises at least one non-radioactive isotope (or ethyl-β-D-glucuronide probe) and at least ethyl-β-D-galactopyranoside (or ethyl-β-D-galactopyranoside probe), the ethyl part of which comprises at least one non-radioactive isotope;
   or at least ethyl-α-mannopyranoside, the ethyl part of which comprises at least one non-radioactive isotope (or ethyl-α-mannopyranoside probe);
b) measuring the amount of labeled ethanol, released in the gas phase, c) comparing the values obtained in step b) with a corresponding standard control value, and
d) deducing therefrom whether the subject is affected by said pathological condition.

According to one embodiment, step a) consists of adding, into a blood sample of the subject, a composition C1-2 or C2-2.

According to one embodiment, step a) consists in adding, into a first blood sample of the subject, a composition C1-2 and, in a second blood sample of the same subject, a composition C2-2.

### Method for Monitoring the Efficacy of a Curative or Preventive Treatment of a Pathological Condition

The present invention also relates to a method for tracking the efficacy of a curative or preventive treatment of a pathological condition in a subject, said pathological condition being selected from the group consisting of pathological conditions involving an inflammatory reaction in response to a lesion or infection of the organism, chronic inflammatory diseases, and acute infections, said method comprising the following steps:
a) adding a composition comprising:
   at least ethyl-β-D-glucuronide, the ethyl part of which comprises at least one non-radioactive isotope;
   at least ethyl-N-acetylglucosamide, the ethyl part of which comprises at least one non-radioactive isotope; and
   at least ethyl-β-D-galactopyranoside, the ethyl part of which comprises at least one non-radioactive isotope;
      in a biological sample of said subject at an instant t during said treatment,
b) measuring the amount of labeled ethanol, released in the gas phase,
c) comparing the values obtained in step b) with a corresponding standard control value and/or with a corresponding value obtained before the start of the treatment or at an instant t′ during the treatment which is prior to the instant t, d) deducing therefrom whether the treatment is effective, and
e) optionally, repeating steps a) to d).

The present invention also relates to a method for tracking the efficacy of a curative or preventive treatment of a pathological condition in a subject, said pathological condition being selected from the group consisting of pathological conditions involving an inflammatory reaction in response to a lesion or infection of the organism, chronic inflammatory diseases, and acute infections, said method comprising the following steps:
a) adding a composition comprising:
   either at least ethyl-β-D-glucuronide, the ethyl part of which comprises at least one non-radioactive isotope (or ethyl-β-D-glucuronide probe) and at least ethyl-β-D-galactopyranoside (or ethyl-β-D-galactopyranoside probe), the ethyl part of which comprises at least one non-radioactive isotope;
   or at least ethyl-α-mannopyranoside, the ethyl part of which comprises at least one non-radioactive isotope (or ethyl-α-mannopyranoside probe);
      in a biological sample of said subject at an instant t during said treatment,
b) measuring the amount of labeled ethanol, released in the gas phase,
c) comparing the values obtained in step b) with a corresponding standard control value and/or with a corresponding value obtained before the start of the treatment or at an instant t′ during the treatment which is prior to the instant t,
d) deducing therefrom whether the treatment is effective, and
e) optionally, repeating steps a) to d).

The pathological condition is in particular as defined above in the “pathological conditions” section. Preferably, the pathological condition is an inflammatory disease, and preferentially a cancer.

The biological sample is a sample from a subject, in particular as defined above in the “biological sample” section. Preferably, the biological sample is a blood, plasma or biopsies sample.

The subject is in particular as defined above in the “subject” section.

The curative or preventive treatment may comprise a curative or preventive treatment of the abovementioned pathological condition and/or a curative or preventive treatment of an inflammation as defined above.

The curative or preventive treatment of a pathological condition involving an inflammatory reaction in response to a lesion or infection of the organism, a chronic inflammatory disease or acute infection is for example a curative or preventive treatment well known to the person skilled in the art for these pathological conditions.

The curative or preventive treatment of the pathological condition may, for example, comprise the surgical removal of the malignant tissue, the administration of a chemotherapy, an immunomodulator, a tritherapy, a suitable antibiotic treatment, or a respiratory assistance.

The features of steps a) and b) are generally as defined for steps a) and b) defined in the “diagnostic method” section.

In the method for monitoring the efficiency of a treatment defined above, step b) comprises measuring the amount, concentration and/or proportion of labeled ethanol in a biological sample of said subject at an instant t during the treatment.

In other words, the measurement is carried out in a biological sample from a sample carried out at instant t.

In step c), the results obtained in step b) are compared with a corresponding standard control value and/or with a corresponding value obtained before the start of said treatment or at an instant during the treatment t′ which is prior to the instant t.

The term “corresponding value obtained before the start of said treatment” is understood to mean the amount, concentration, and/or proportion of labeled ethanol measured in a biological sample of said subject, said sample having been taken before the beginning of the treatment.

The term “corresponding value obtained at an instant during the treatment which is prior to instant t” is understood to mean the quantity, concentration, and/or proportion of corresponding labeled ethanol in a biological sample of said subject, said sample having been taken after the start of the treatment, but before instant t.

In step d), it is deduced therefrom whether the treatment is effective.

The treatment is for example effective if the measured amount of labeled ethanol decreases or disappears.

For example, the treatment is effective if its quantity, concentration, and/or proportion measured at instant t is less than a corresponding value obtained before the start of said treatment or at an instant t′ during the treatment which is prior to instant t.

For example, the treatment is ineffective if the quantity, concentration, and/or proportion of labeled ethanol measured at instant t is greater than a corresponding value obtained before the start of said treatment or at an instant t′ during the treatment which is prior to instant t.

If the treatment is ineffective, the method may comprise the administration of a new treatment or a complementary treatment.

The method may comprise a step e) wherein steps a) to d) are repeated, in particular whether the treatment is effective or not.

Steps a) to d) can be repeated once, twice, three times or at least three times.

Steps a) to d) can for example be repeated until the subject has recovered or the pathological condition has stabilized.

The period between two repetitions may for example be at least one week, at least two weeks, at least three weeks, at least four weeks, at least two months, at least three months, at least four months, at least five months, six months, or at least six months, for example one year.

The method for monitoring the efficacy of a curative or preventive treatment can be carried out each time a new treatment or complementary treatment is initiated.

A subject is considered to have recovered when the ethanol value measured according to step b) is included in the standard deviation relative to the average of the healthy subjects over all of the kinetic points (as explained above in the paragraph concerning the diagnostic method).

### Method for Monitoring the Evolution of a Pathological Condition

The present invention also relates to a method for tracking the course of a pathological condition in a subject, said pathological condition being selected from the group consisting of pathological conditions involving an inflammatory reaction in response to a lesion or infection of the organism, chronic inflammatory diseases, and acute infections, said method comprising the following steps:
a) adding, into a biological sample of said subject, at an instant t1, a composition comprising:
   at least ethyl-β-D-glucuronide, the ethyl part of which comprises at least one non-radioactive isotope;
   at least ethyl-N-acetylglucosamide, the ethyl part of which comprises at least one non-radioactive isotope; and
   at least ethyl-β-D-galactopyranoside, the ethyl part of which comprises at least one non-radioactive isotope;
b) measuring the amount of labeled ethanol, released in the gas phase, for the sample at instant t1,
c) adding, in a biological sample of said subject, at an instant t2 separated in time from instant t1, the composition as defined in step a), d) measuring the amount of labeled ethanol, released in the gas phase, for the sample at instant t2,
e) comparing the values obtained in steps b) and d) at instants t1 and t2 respectively,
f) deducing therefrom whether the pathological condition changes favorably, and
g) optionally, repeating steps a) to f).

The present invention also relates to a method for tracking the course of a pathological condition in a subject, said pathological condition being selected from the group consisting of pathological conditions involving an inflammatory reaction in response to a lesion or infection of the organism, chronic inflammatory diseases, and acute infections,

said method comprising the following steps:
a) adding, into a biological sample of said subject, at an instant t1, a composition comprising:
   either at least ethyl-β-D-glucuronide, the ethyl part of which comprises at least one non-radioactive isotope (or ethyl-β-D-glucuronide probe) and at least ethyl-β-D-galactopyranoside (or ethyl-β-D-galactopyranoside probe), the ethyl part of which comprises at least one non-radioactive isotope;
   or at least ethyl-α-mannopyranoside, the ethyl part of which comprises at least one non-radioactive isotope (or ethyl-α-mannopyranoside probe);
b) measuring the amount of labeled ethanol, released in the gas phase, for the sample at instant t1,
c) adding, in a biological sample of said subject, at an instant t2 separated in time from instant t1, the composition as defined in step a), d) measuring the amount of labeled ethanol, released in the gas phase, for the sample at instant t2,
e) comparing the values obtained in steps b) and d) at instants t1 and t2 respectively,
f) deducing therefrom whether the pathological condition changes favorably, and
g) optionally, repeating steps a) to f).

The pathological condition is in particular as defined above in the “pathological conditions” section. Preferably, the pathological condition is an inflammatory disease, and preferentially a cancer.

The biological sample is a sample from a subject, in particular as defined above in the “biological sample” section. Preferably, the biological sample is a blood, plasma or biopsies sample.

The subject is in particular as defined above in the “subject” section.

The features of steps a) to d) are generally as defined for steps a) and b) defined in the “diagnostic method” section.

In the method for monitoring the course of a pathological condition defined above, steps b) and d) consist of measuring the amount, concentration, and/or proportion of labeled ethanol in a biological sample of the subject at two instants t1 and t2 separated in time.

In other words, the measurement is carried out in a biological sample coming from a sample carried out at an instant t1, as well as in a biological sample coming from a sample taken at the instant t2, the instant t1 being prior to the instant t2. The time interval between t1 and t2 is for example be at least one week, at least two weeks, at least three weeks, at least four weeks, at least two months, at least three months, at least four months, at least five months, six months, or at least six months, for example one year.

In step e), the results obtained in step a), at instant t1 and at instant t2 are compared with each other.

In step f), it is deduced therefrom whether the pathological condition is progressing favorably.

The pathological condition evolves favorably, for example if the amount of released ethanol decreases between instant t1 and t2.

The pathological condition is not progressing favorably, for example if the amount of ethanol released is equal or increases between instant t1 and t2.

The progression of the disease is considered to be highly favorable when the ethanol value measured for the sample is included in the standard deviation relative to the average of the values for the healthy subjects over all of the kinetic points (as explained above in the paragraph concerning the diagnostic method).

The progression of the disease is considered to be highly unfavorable when the ethanol value measured for the sample is included in the standard deviation or greater than the standard deviation relative to the average of the values for the diseased subjects over all of the kinetic points (as explained above in the paragraph concerning the diagnostic method). This value is at least 1.3 times greater than the average of the values measured for healthy subjects on at least one of the kinetic points.

The method may comprise a step g) wherein steps a) to f) are repeated, in particular whether the pathological condition is progressing favorably or not.

Steps a) to f) can be repeated once, twice, three times or at least three times.

Steps a) to f) can for example be repeated until the subject has recovered or the pathological condition has stabilized.

Steps a) to d) can be repeated at regular or irregular time intervals. The period between two repetitions may for example be at least one week, at least two weeks, at least three weeks, at least four weeks, at least two months, at least three months, at least four months, at least five months, six months, or at least six months, for example one year.

Method of Categorizing a Subject Suffering or at Risk of Suffering from a Pathological Condition

The present invention also relates to a method for categorizing a subject suffering from a pathological condition in a category C-1 of subjects suffering from said pathological condition at a degree of severity or in a category C-2 of subjects suffering from said pathological condition at another degree of severity, said pathological condition being selected from the group consisting of pathological conditions involving an inflammatory reaction in response to a lesion or infection of the organism, chronic inflammatory diseases and acute infections, said method comprising the following steps:
a) adding, in a biological sample of said subject, a composition comprising:
   at least ethyl-β-D-glucuronide, the ethyl part of which comprises at least one non-radioactive isotope;
   at least ethyl-N-acetylglucosamide, the ethyl part of which comprises at least one non-radioactive isotope; and
   at least ethyl-β-D-galactopyranoside, the ethyl part of which comprises at least one non-radioactive isotope;
b) measuring the amount of labeled ethanol, released in the gas phase, c) comparing the values obtained in step b) with a corresponding standard control value, and
d) deducing therefrom whether the subject is in the category C-1 or in the category C-2.

The present invention also relates to a method for categorizing a subject suffering from a pathological condition in a category C-1 of subjects suffering from said pathological condition at a degree of severity or in a category C-2 of subjects suffering from said pathological condition at another degree of severity, said pathological condition being selected from the group consisting of pathological conditions involving an inflammatory reaction in response to a lesion or infection of the organism, chronic inflammatory diseases and acute infections,

said method comprising the following steps:
a) adding, in a biological sample of said subject, a composition comprising:
   either at least ethyl-β-D-glucuronide, the ethyl part of which comprises at least one non-radioactive isotope (or ethyl-β-D-glucuronide probe) and at least ethyl-β-D-galactopyranoside (or ethyl-β-D-galactopyranoside probe), the ethyl part of which comprises at least one non-radioactive isotope;
   or at least ethyl-α-mannopyranoside, the ethyl part of which comprises at least one non-radioactive isotope (or ethyl-α-mannopyranoside probe);
b) measuring the amount of labeled ethanol, released in the gas phase,
c) comparing the values obtained in step b) with a corresponding standard control value, and
d) deducing therefrom whether the subject is in the category C-1 or in the category C-2.

The pathological condition is in particular as defined above in the “pathological conditions” section. Preferably, the pathological condition is an inflammatory disease, and preferentially a cancer.

The biological sample is a sample from a subject, in particular as defined above in the “biological sample” section. Preferably, the biological sample is a blood, plasma or biopsies sample.

The subject is in particular as defined above in the “subject” section.

The features of steps a) to c) are generally as defined for steps a) and b) defined in the “diagnostic method” section.

### Kit and Use for Diagnosis

The present invention also relates to a kit for the diagnosis of a pathological condition selected from the group consisting of pathological conditions involving an inflammatory reaction in response to a lesion or infection of the organism, chronic inflammatory diseases and acute infections,

said kit comprising a composition comprising:
at least ethyl-β-D-glucuronide, the ethyl part of which comprises at least one non-radioactive isotope;
at least ethyl-N-acetylglucosamide, the ethyl part of which comprises at least one non-radioactive isotope; and
at least ethyl-β-D-galactopyranoside, the ethyl part of which comprises at least one non-radioactive isotope.

The present invention also relates to a kit for the diagnosis of a pathological condition selected from the group consisting of pathological conditions involving an inflammatory reaction in response to a lesion or infection of the organism, chronic inflammatory diseases and acute infections, said kit comprising a composition comprising:
either at least ethyl-β-D-glucuronide, the ethyl part of which comprises at least one non-radioactive isotope (or ethyl-β-D-glucuronide probe) and at least ethyl-β-D-galactopyranoside (or ethyl-β-D-galactopyranoside probe), the ethyl part of which comprises at least one non-radioactive isotope;
or at least ethyl-α-mannopyranoside, the ethyl part of which comprises at least one non-radioactive isotope (or ethyl-α-mannopyranoside probe).

The present invention also relates to a kit for the diagnosis of a pathological condition selected from the group consisting of pathological conditions involving an inflammatory reaction in response to a lesion or infection of the organism, chronic inflammatory diseases and acute infections,

said kit comprising a composition C1 or C2 as defined above.

The present invention also relates to a use of the aforementioned kit for the diagnosis of a pathological condition selected from the group consisting of pathological conditions involving an inflammatory reaction in response to a lesion or infection of the organism, chronic inflammatory diseases and acute infections.

### FIGURES

FIG. 1 shows the calibration straight line for the detection of β-glucuronidase in the presence of the probe D₅-ethyl-β-D-glucuronide and performance of the analytical method GC-MSMS (LOD, LOQ and R²).

FIG. 2 shows the calibration straight line for the detection of β-glucuronidase in the presence of the para-nitrophenol glucuronide and performance of the UV detection method (LOD, LOQ and R²).

FIG. 3 shows the kinetics of ethanol-D₅ detection released into the headspace of the plasmas of patients after adding the probe D₅-ethyl-β-D-glucuronide.

FIG. 4 shows the kinetics of ethanol-D₅ detection released into the headspace of the plasmas of patients having tumors of different sizes after adding the probe D₅-ethyl-β-D-glucuronide.

FIG. 5 shows the detection of ethanol-D₅ in the headspace of mouse tissue samples as a function of the concentration of the added probe Ds-ethyl-β-D-glucuronide (10⁻⁹ M).

FIG. 6 shows the amount of ethanol-D₅ and ethanol-¹³C-D₅ released after hydrolysis of each probe in the presence of their target enzyme. β-Glucuronidase (enzyme 1) and D₅-ethyl-β-D-glucuronide (probe 1). N-Acetyl-glucosaminidase (enzyme 2) and ¹³C-D₅-ethyl-N-acetylglucosamine (probe 2).

FIG. 7 shows the amount of ethanol-D₅ and ethanol-¹³C-D₅ released after hydrolysis of each probe in the presence of their partner enzyme. N-Acetyl-glucosaminidase (enzyme 2) and D₅-ethyl-β-D-glucuronide (probe 1). β-Glucuronidase (enzyme 1) and ¹³C-D₅-ethyl-N-acetylglucosamine (probe 2).

FIG. 8 shows the amount of ethanol-D₅ and ethanol-¹³C-D₅ released when the two probes are placed in the presence of one or the other of the enzymes β-Glucuronidase (enzyme 1), D₅-ethyl-β-D-glucuronide (probe 1) and ¹³C-D₅-ethyl-N-acetylglucosamine (probe 2). N-Acetyl-glucosaminidase (enzyme 2) and D₅-ethyl-β-D-glucuronide (probe 1) and ¹³C-D₅-ethyl-N-acetylglucosamine (probe 2).

FIG. 9a shows the amount of ethanol-D₅ released after hydrolysis of D₅-ethyl-β-D-glucuronide targeting β-glucuronidase (n=4 healthy patients, n=3 patients with breast cancer, n=3 patients with lung cancer). FIG. 9b shows the amount of ethanol-D4 released after hydrolysis of D₄-ethyl-1-α-L-fucopyranoside targeting α-L-fucosidase (n=2 healthy patients, n=1 patient with breast cancer, n=1 patient with lung cancer). FIG. 9c shows the amount of ethanol-¹³CD₅ released after hydrolysis of the ¹³CD₅-ethyl-N-acetylglucosamine targeting the N-acetyl-glucosaminidase (n=4 healthy patients, n=3 patients with breast cancer, n=3 patients with lung cancer). FIG. 9d shows the amount of ethanol-D2 released after hydrolysis of D2-ethyl-β-D-galactopyranoside targeting β-D-galactosidase (n=4 healthy patients, n=3 patients with breast cancer, n=3 patients with lung cancer).

FIG. 10a shows the amount of ethanol-D₅ exhaled by animals after injection of 10 pg·kg⁻¹ of D₅-ethyl-β-D-glucuronide. FIG. 10b shows the amount of ethanol-D4 exhaled by the animals after injection of 100 pg·kg⁻¹ D4-ethyl-1-α-L-fucopyranoside. FIG. 10c shows the amount of ethanol-¹³CD₅ exhaled by animals after injection of 10 pg·kg⁻¹ of ¹³CD₅-ethyl-N-acetylglucosamine. FIG. 10d shows the amount of ethanol-D₂ exhaled by animals after injection of 1 pg·kg⁻¹ of D₂-ethyl-β-D-galactopyranoside.

FIG. 11 shows the calibration straight lines having the signal intensity of ethanol-D₅ depending on the activity (U·L⁻¹) of α-mannosidase in the presence of the probe D₅-ethyl-α-mannopyranoside (10³M) and the performance of the VOC probe concept for the detection of this enzyme (LOD and R²). The straight lines were plotted for three different kinetic points (2, 4 and 7 h of reaction). Each point was carried out in duplicate. FIG. 11a corresponds to the kinetic point 2 h to 2 h30 min, FIG. 11b corresponds to the kinetic point 4 h to 4 h30 min and FIG. 11c corresponds to the kinetic point 7 h to 7 h30 min.

FIG. 12 shows the calibration straight lines having the signal intensity of ethanol-D2 depending on the activity (U·L⁻¹) of β-galactosidase in the presence of the probe D₂-ethyl-β-D-galactopyranoside (10⁴ M) and the performance of the VOC probe concept for the detection of this enzyme (LOD and R²). The straight lines were plotted for three different kinetic points (2, 4 and 7 h of reaction). Each point was carried out in duplicate. FIG. 12a corresponds to the kinetic point 2 h to 2 h30 min, FIG. 12b corresponds to the kinetic point 4 h to 4 h30 min and FIG. 12c corresponds to the kinetic point 7 h to 7 h30 min.

FIG. 13 shows the calibration straight lines having the signal intensity of ethanol-¹³CD₅ depending on the activity (U·L⁻¹) of N-acetyl-glucosaminidase in the presence of the probe ¹³C-D₅-ethyl-N-acetylglucosamide (10³M) and the performance of the VOC probe concept for the detection of this enzyme (LOD and R²). The straight lines were plotted for three different kinetic points (2, 4 and 7 h of reaction). Each point was carried out in duplicate. FIG. 13a corresponds to the kinetic point 2 h to 2 h30 min, FIG. 13b corresponds to the kinetic point 4 h to 4 h30 min and FIG. 13c corresponds to the kinetic point 7 h to 7 h30 min.

FIG. 14a shows the amount of ethanol-D₅ detected above the biopsies in the presence of 10⁻⁷ M of D₅-ethyl-β-D-glucuronide. FIG. 14b shows the amount of ethanol-D detected above the biopsies in the presence of 10⁻⁴ M of D₄-ethyl-1-α-L-fucopyranoside. FIG. 14c shows the amount of ethanol-¹³CD₅ detected above the biopsies in the presence of 10⁻⁸ M of ¹³CD₅-ethyl-N-acetylglucosamine. FIG. 14d shows the amount of ethanol-D₂ detected above the biopsies in the presence of 10⁻⁶ M of D₂-ethyl-β-D-galactopyranoside.

FIG. 15 relates to the kinetics of hydrolysis of probes based on VOCs (n=7 healthy patients; solid line=blank; dotted line=mean of the responses of blood samples reserved for 30 min at room temperature before treatment: healthy-T0; dashed line=average of the responses of the blood samples reserved for 5 h at 4° C. before treatment: healthy-T2). FIG. 15a shows the amount of ethanol-D₅ released after hydrolysis of D₅-ethyl-β-D-glucuronide targeting β-glucuronidase. FIG. 15b shows the amount of ethanol-D released after hydrolysis of D₄-ethyl-1-α-L-fucopyranoside targeting α-L-fucosidase. FIG. 15c shows the amount of ethanol-¹³CD₅ released after hydrolysis of the ¹³CD₅-ethyl-N-acetylglucosamine targeting the N-acetyl glucosaminidase. FIG. 15d shows the amount of ethanol-D₂ released after hydrolysis of D₂-ethyl-β-D-galactopyranoside targeting β-D-galactosidase. FIG. 15e shows the amount of ethanol-D₅ released after hydrolysis of D₅-ethyl-α-mannopyranoside targeting α-mannosidase. FIG. 15f shows the amount of ethanol-D4 released after hydrolysis of the D4-ethyl-α-D-glucuronide targeting α-D-glucosidase.

FIG. 16 relates to the prognostic efficiency of the probes based on VOCs during SARS-COV-2 infection (n=10 slightly symptomatic patients: group A, n=10 strongly symptomatic patients: group B, n=10 strongly symptomatic patients admitted into intensive care: group C). FIG. 16a shows the amount of ethanol-D₅ released after hydrolysis of D₅-ethyl-β-D-glucuronide targeting β-glucuronidase. FIG. 16b shows the amount of ethanol-D released after hydrolysis of D₄-ethyl-1-α-L-fucopyranoside targeting α-L-fucosidase. FIG. 16c shows the amount of ethanol-¹³CD₅ released after hydrolysis of the ¹³CD₅-ethyl-N-acetylglucosamine targeting N-acetyl glucosaminidase. FIG. 16d shows the amount of ethanol-D₂ released after hydrolysis of D₂-ethyl-β-D-galactopyranoside targeting β-D-galactosidase. FIG. 16e shows the amount of ethanol-D₅ released after hydrolysis of D₅-ethyl-α-mannopyranoside targeting α-mannosidase. FIG. 16f shows the amount of ethanol-D4 released after hydrolysis of the D4-ethyl-α-D-glucuronide targeting α-D-glucosidase.

FIG. 17 relates to the progression of the activity of enzymatic markers detected via the probes based on VOCs during SARS-COV-2 infection (n=10 slightly symptomatic patients: group A, n=10 strongly symptomatic patients: group B, n=10 strongly symptomatic patients admitted into intensive care: group C). FIG. 17a shows the amount of ethanol-D₅ released after hydrolysis of D₅-ethyl-β-D-glucuronide targeting β-glucuronidase. FIG. 17b shows the amount of ethanol-D4 released after hydrolysis of D₄-ethyl-1-α-L-fucopyranoside targeting α-L-fucosidase. FIG. 17c shows the amount of ethanol-¹³CD₅ released after hydrolysis of the ¹³CD₅-ethyl-N-acetylglucosamine targeting N-acetyl glucosaminidase. FIG. 17d shows the amount of ethanol-D₂ released after hydrolysis of D₂-ethyl-β-D-galactopyranoside targeting β-D-galactosidase. FIG. 17e shows the amount of ethanol-D₅ released after hydrolysis of D₅-ethyl-α-mannopyranoside targeting α-mannosidase. FIG. 17f shows the amount of ethanol-D4 released after hydrolysis of the D4-ethyl-α-D-glucuronide targeting α-D-glucosidase. FIG. 18 shows the calibration straight lines having the signal intensity of ethanol-D4 depending on the activity (U·L⁻¹) of α-fucosidase in the presence of the probe D-ethyl-1-α-L-fucopyranoside (10⁻³M) and the performance of the VOC probe concept for the detection of this enzyme (LOD and R²). The straight lines were plotted for three different kinetic points (2, 4 and 7 h of reaction). Each point was carried out in duplicate. FIG. 18a corresponds to the kinetic point 2 h to 2 h30 min, FIG. 18b corresponds to the kinetic point 4 h to 4 h30 min and FIG. 18c corresponds to the kinetic point 7 h to 7 h30 min.

### EXAMPLES

### Example 1: Optimization of the Protocol for Detecting Tumor Enzyme Markers in Blood Samples; Validation with a Probe of the Cocktail

An ex vivo study aims to demonstrate the relevance of the probes of the invention to diagnose the most widespread solid cancers from a simple patient blood sample was carried out.

This preliminary study was carried out with one of the probes of the cocktail, the probe D₅-ethyl-β-D-glucuronide (probe (1) defined above), targeting β-glucuronidase and releasing, after hydrolysis, a labeled VOC, ethanol-D₅.

In order to detect the enzyme in plasma samples, the optimal conditions for hydrolysis of the β-glucuronidase within this matrix were studied. A sample of plasma taken from a healthy patient was used to optimize all of the conditions. The β-glucuronidase of E. coli was used as an enzymatic model. Different parameters were tested such as the buffer used (experiment 1), the reaction volume (experiment 2), the optimum probe concentration to be added into the sample (experiment 3), the impact of the freezing/thawing of the plasmas on the activity of the enzyme (experiment 4) and finally the impact of the plasma proteases on the activity of the enzyme (experiment 5). All of these experiments are summarized in Table 1.

TABLE 1
Optimization of optimal conditions for hydrolysis of the β-glucuronidase in plasma.

| Experiments | 1 | 2 | 3 | 4 | 5 |
|---|---|---|---|---|---|
| Buffer (pH) | Phosphate | Phosphate | Phosphate | Phosphate | Acetate |
| | buffer (pH 7) | buffer (pH 7) | buffer (pH 7) | buffer (pH 7) | buffer (pH 5) |
| | Acetate | | Acetate | | |
| | buffer (pH 5) | | buffer (pH 5) | | |
| Reaction volume | 50-25 | 50-25 | 50-25 | 50-25 | 100-25 |
| (pL)-plasma | | 100-25 | | | |
| volume (pL) | | 200-25 | | | |
| [Probe] (mol · L⁻¹) | 5.10⁻⁵ | 5.10⁻⁵ | 10⁻⁴ | 5.10⁻⁵ | 5.10⁻⁵ |
| | | | 5.10⁻⁵ | | |
| | | | 10⁻⁵ | | |
| Freezing/thawing | / | / | / | 3 6-day | / |
| | | | | freezing/ | |
| | | | | thawing | |
| | | | | cycles. | |
| Protease inhibitor | / | / | / | / | Without, |
| | | | | | 1 X or 2X |
| β-glucuronidase (mol · L⁻¹) | 10⁻⁹ | 10⁻⁹ | / | 10⁻⁹ | 10⁻⁹ |

According to this experimental plan, the optimal plasma probe hydrolysis conditions were determined, as explained hereinafter:

Thus, a sample of 4-5 mL of blood is taken from patients at the time of their first consultation with the attending oncologist.

The plasma (approximately 2 μL) is extracted and stored at −20° C. until analysis. An aliquot of 25 μL of plasma is sampled and inserted into a 2 μL vial with an insert. This biological sample is added to 50 μL of acetate buffer solution (pH 5), protease inhibitor (1×) and 25 μL probe D₅-ethyl-β-D-glucuronide at 2.10⁴ M. After hermetic closure and agitation to the vortex of the vial, an SPME (SPME in semi-polar phase of the Car/PDMS, 1 cm long and 75 μm thick type with a 24 ga needle; Supelco) of the gas phase above the sample of a duration of 30 mi is carried out after 2 h, 4 h and 7 h of reaction. Each kinetic point requires a vial reaction, and is carried out in duplicate. The VOCs trapped on the SPME fiber are then analyzed by G-TQMS. GC-TMS detection of the ethanol isotopes (thus including ethanol-D₅) is carried out with gas chromatography coupled to a triple quadrupole equipped with an E1 source (TSQ 9000, Thermo Fisher Scientific). The system is controlled by the software TSQ 9000 and the results are analyzed with the Xcalibur software (Thermo Fisher Scientific).

The analysis conditions are summarized in table 2.

TABLE 2
Settings GC and TQ of the method enabling
the analysis of the VOCs.

| | GC-MS Settings | Method 3 |
|---|---|---|
| GC | Injector temperature (° C.) | 280 |
| Settings | Septum purge and gas saver (mL · min−¹) | 5-20 |
| | Flow rate of the gas-helium (mL · min¹) | 1.4 |
| | Split/Splitless | Splitless |
| | GC Program | 0-7 min: 90° C. |
| TQ | Mode | SRM |
| Settings | Ionization energy (eV) | 70 |
| | Scan speed (scans · s¹) | 20 |
| | Injector temperature, transfer | 280 - 260 - 260 |
| | line and source (° C.) | |

For each control measurement, the absence of isotopes of ethanol must be verified in all the vials used for each study with VOC probes. Thus, an SPME sample of 10 min of the VOCs is carried out before the start of the experiment, then analyzed by GC-TQMS.

All the so-called “tracked exogenous VOC-polluted” vials are not used.

For detection in SRM mode (Selected Reaction Monitoring), the transitions of each labeled VOC must be previously optimized. Ethanol-D₅ is followed with the transitions 51>33 and 51>49, different from those of unlabeled ethanol that can interfere with the analysis (transitions 46>31 and 46>45).

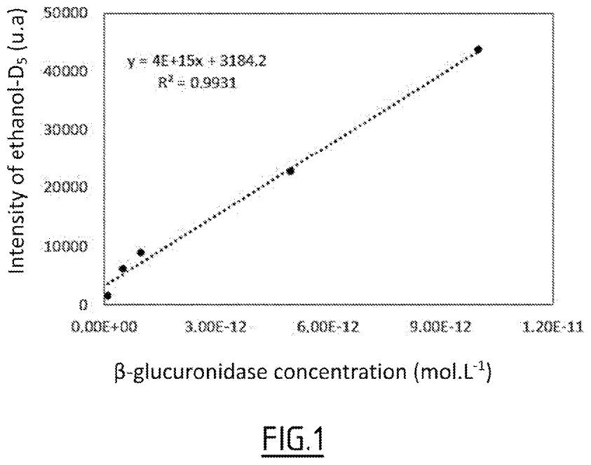

The limit of detection (LOD) of a model enzyme (β-glucuronidase of E. coli; liquid solution 50% glycerol, 140 U·mL⁻¹; Sigma Aldrich) in plasma matrix in the presence of probe D₅-ethyl-β-D-glucuronide was then determined. For this, a plasma sample for which the activity of endogenous β-glucuronidase was not detectable, was added to enzyme concentrations ranging from 1.10¹¹ to 1.10⁻¹³ mol·L ⁻¹. The amount of ethanol-D₅ released was monitored before adding probe and then after 2.5, 4.5 and 7.5 h of reaction at 30° C. FIG. 1 shows the calibration straight line allowing the β-glucuronidase detection after 7.5 h of reaction.

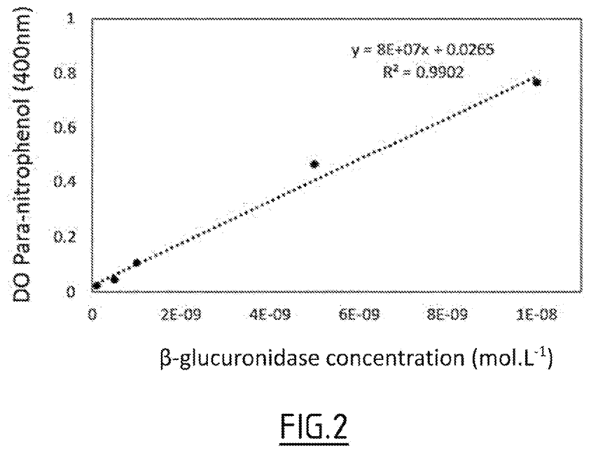

The limit of detection of the enzyme with the probe is between 1.10¹³ mol·L⁻¹ (signal to noise 9) and 5×10¹³ mol·L⁻¹ (signal to noise 37), which leads to a minimum quantity of detectable enzyme equal to 1.10⁻¹⁸ mol. This LOD is 100 times lower than the threshold value obtained according to the reference detection method of this same enzyme (Li et al., 2018, Biotechnology Letters, 40, 11 1-118; Xiao et al., 2020, RSC Advances, 10, 22966). This method corresponds to the hydrolysis of para-nitrophenol glucuronide. It was carried out in acetate buffer (pH>10), followed by UV detection at 400 nm of the para-nitrophenol (FIG. 2).

Finally, compared to the literature, the approach according to the present invention has proved to be 100 to 1000 times more sensitive than the very recent strategies involving fluorescent probes like the para-nitrophenol glucuronide (Huo et al. 2018, Sensors and Actuators B: Chemical, 262, 508-515; Guo et al. 2019, Sensors and Actuators B: Chemical, 295, 1-6). It is also 10 times more sensitive than the most sensitive proteomic analyses (Selevsek et al. 2011, Proteomics, 11, 1135-1147).

After having optimized the strategy for detecting β-glucuronidase and evaluated the performance of the VOC probe coupled to the analytical method, a clinical study on plasma samples was carried out. The associated clinical protocol was established by the CIC (PU/PH R. Robert, INSERM 1402, CHU Poitiers) and then subjected to the evaluation of the Ethics Committee of CHU Poitiers hospital.

Blood samples were taken by the caregiver associated with the oncological service, then collected and pretreated with the CIC. This study included samples of plasma taken from 11 patients suffering from breast cancer and 11 patients suffering from lung cancer.

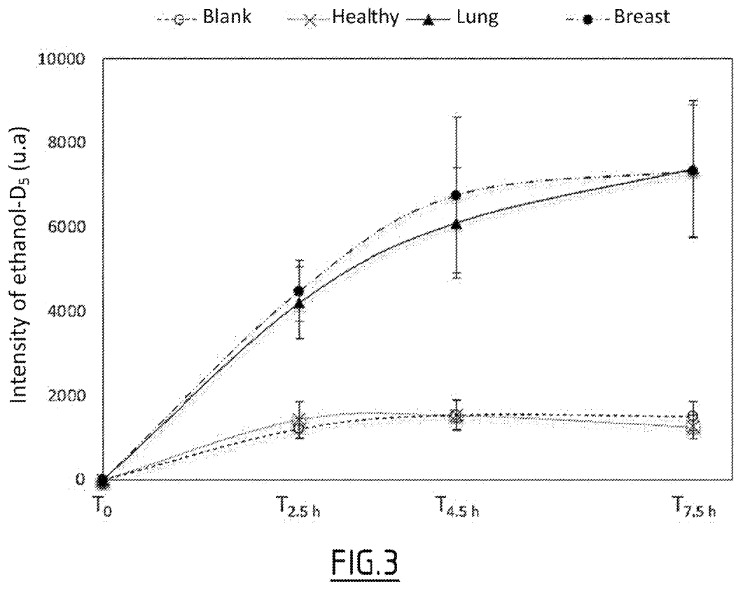

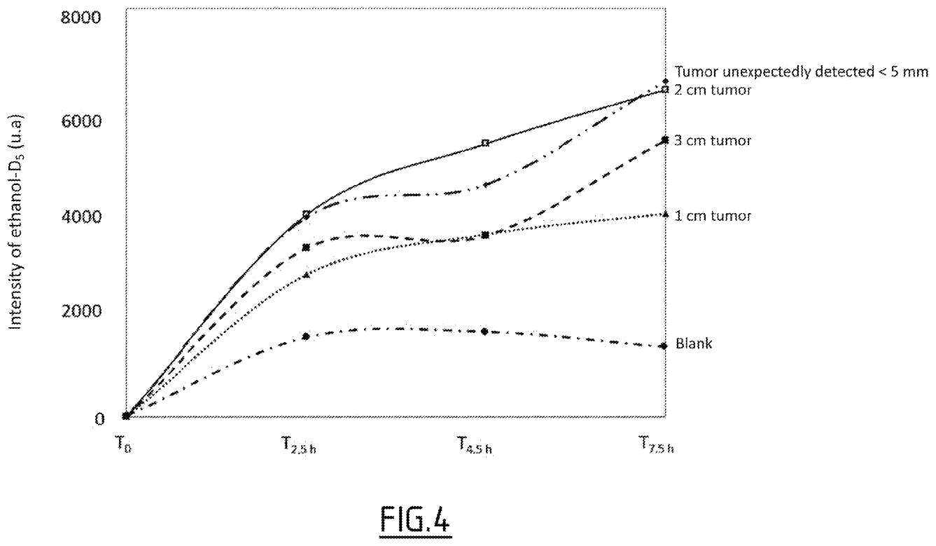

These patients were diagnosed, but had at the time of sampling not yet received treatment. Their response was compared to that obtained from the plasma of 4 healthy patients. The results were compared to an experimental blank wherein the probe was added, but the enzyme was not present (FIG. 3).

After adding probe, the amount of ethanol-D₅ released above the samples of patients with cancer was significantly higher compared to the blanks and to the healthy patient's plasma. This test therefore makes it possible to discern healthy patients from cancer patients.

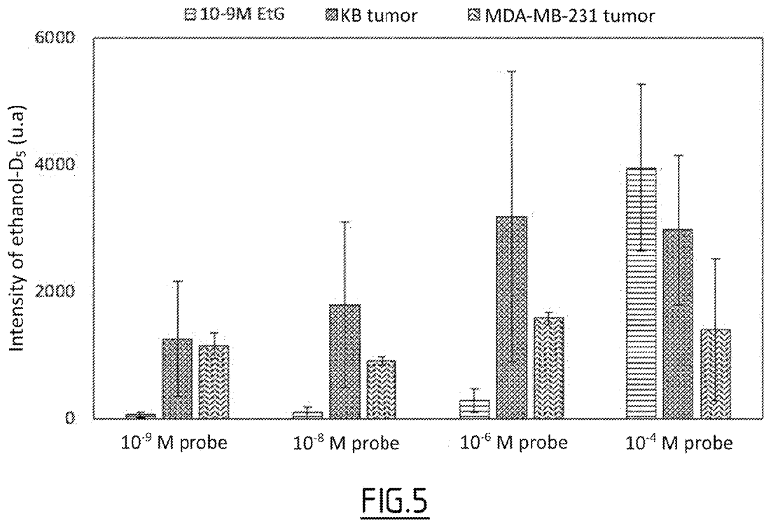

It appears that the amount of enzyme present in the plasmas of the patients is not positively correlated to the size of the tumor (FIG. 4). This being, other factors such as the aggressiveness of cancer or its stage of evolution can be studied in order to confirm a correlation between these physiopathological parameters and the activity of the plasma enzyme.

In addition, according to the clinical and biological data of patients, it has been demonstrated that our strategy made it possible to detect tumors of subcentimeter size, such as one discovered unexpectedly in a patient (curve with diamonds, FIG. 4). Such a result demonstrates the sensitivity of the VOC probes to detect a tumor, even though imaging was not able to confirm this diagnosis.

### Example 2: Optimization of the Protocol for Detecting Tumor Enzyme Markers in Tissue Samples (Biopsies); Validation with a Probe of the Cocktail

Here, the objective is to develop a protocol making it possible to detect the target enzymes in the extracellular environment of tumor cells, whereas they should be absent, or have limited activity, in the environment of healthy cells.

This protocol was first developed in order to detect β-glucuronidase with the corresponding probe of the cocktail, D₅-ethyl-β-D-glucuronide.

It is first necessary to determine the probe concentration to be added to the tissue to detect only the enzyme present in the extracellular medium.

Thus, tests during which the tumoral and healthy biopsies were placed in the presence of decreasing concentrations of D₅-ethyl-β-D-glucuronide were carried out. The probe concentration retained corresponded to the concentration for which no response of healthy tissues was obtained, whereas an enzymatic response was observed on cancer tissues.

In the context of this work, two tumor biopsies, taken on mouse models, were studied (MDA-MB-231: human breast cancer cells, KB: human nasopharynx cancer cells). Two mouse liver biopsies were used as healthy tissues. Probe solutions at 10⁻⁷, 10⁻⁶, 10⁻⁴ and 10⁻² M were prepared in a PBS buffer (137 mM NaCl, 2.7 mM KCl, 10 mM Na₂HPO₄, 1.8 mM KH₂PO₄, pH 7.4).

The biopsies were rinsed with 500 μL of PBS and then placed in a vial with an insert of 2 mL. A volume of 297 μL of PBS was added to the vial. The volatile molecules present in the headspace of the vial were trapped for 30 min in order to ensure that no ethanol-D₅ was detected. Then 3 μL of probe at 10⁷, 10⁶, 10⁴ or 10² M were added (final probe concentration in the vial of 10⁹, 10⁸, 10⁶ or 10⁴ M). After hermetic closure and vortex agitation, the volatile compounds were preconcentrated on SPME fiber (Car/PDMS, Supelco) for 30 min and then analyzed by GC-TQMS (analysis conditions described in Table 2).

For each control measurement, the absence of isotopes of ethanol must be verified in all the vials used for the study. Thus, an SPME sample of 10 min of the VOCs is carried out before the start of the experiment, then analyzed by GC-TQMS. All the so-called “tracked exogenous VOC-polluted” vials are not used.

Despite significant standard deviations for tumor biopsies, it clearly appears that a probe concentration of less than 10⁻⁴ M makes it possible to discern healthy tissues from cancerous tissues (FIG. 5). Indeed, no response in ethanol-D₅ was detected on healthy tissues for probe concentrations equal to 10⁻⁹, 10⁸ and 10⁶ M, whereas at these concentrations, release of this isotope was detected for cancer tissues. Such a result suggests that at these concentrations the probe did not passively penetrate the cells, and was therefore not hydrolyzed by the lysosomal β-glucuronidase of tumor cells and healthy cells. On the other hand, the probe was indeed hydrolyzed by the β-glucuronidase present in the extracellular matrix of cancer tissues. Thus, after optimization of the dose to be added to the medium for each probe belonging to the cocktail, it is possible to map the activities of the target enzymes directly to the tissue biopsies.

### Example 3: Preliminary Tests with a Cocktail of Four VOC-Based Probes According to the Invention

A second probe, ¹³C-D₅-ethyl-N-acetylglucosamine (probe (2) as defined above) targeting the N-acetyl-glucosaminidase was studied. In order to differentiate the ethanol released after hydrolysis of this probe from the ethanol released after hydrolysis of D₅-ethyl-β-D-glucuronide, a targeted analysis method using GC-MS/MS (SRM mode) was developed and the transitions of the isotope ¹³C-D₅ were optimized (transitions of ethanol-¹³C-D₅: 52>34 and 52>50).

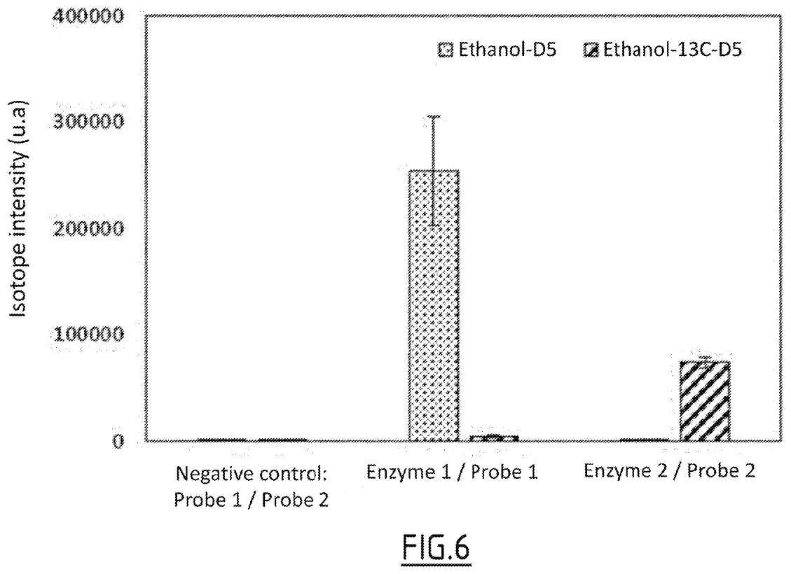

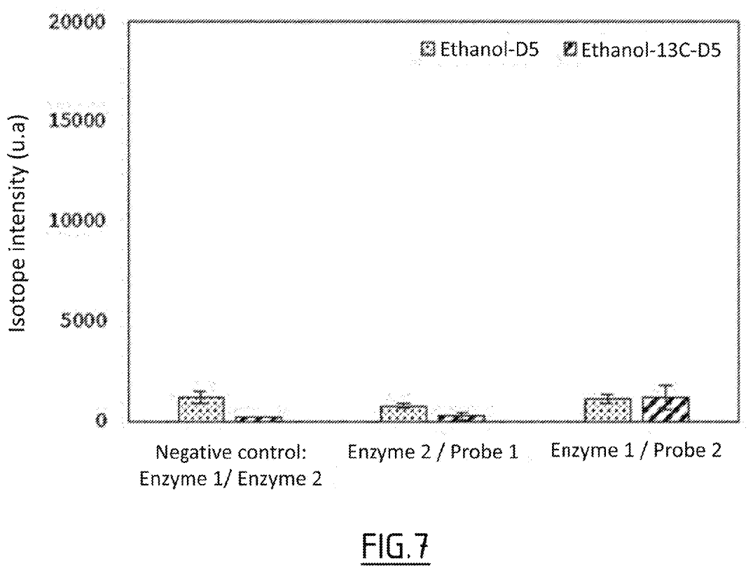

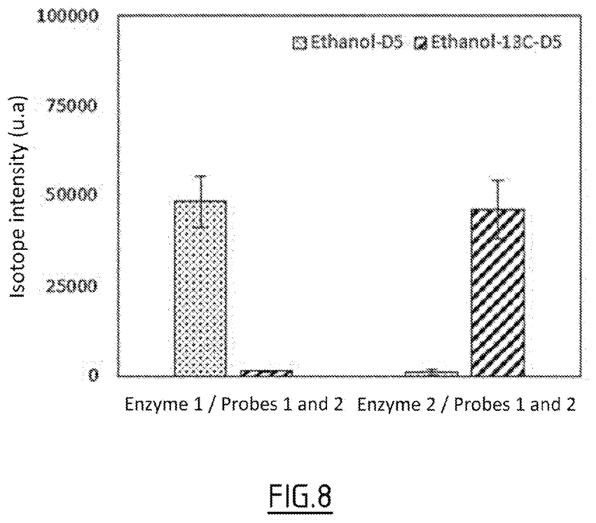

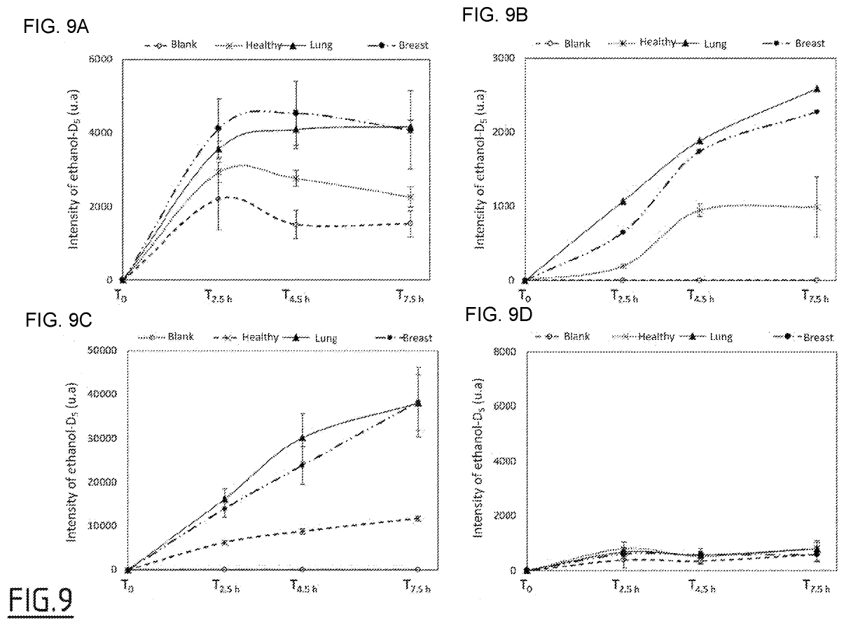

The detection performances of this compound in the presence of ethanol and ethanol-D₅ were then verified. It was then demonstrated that in the presence of ethanol and ethanol-D₅, the specificity and sensitivity of the detection of ethanol-¹³C-D₅ are in no way altered. The detection ranges of ethanol-D₅ and ethanol-¹³C-D₅ were then carried out under SPME sampling conditions developed for biological samples (30 min of sampling at 30° C.). The two isotopes were present in mixtures in solution. The limits of detection obtained are respectively 1.10⁹ M and 5.10⁹ M. The SPME-GC-TQMS method therefore allows a sharp differentiation of the two isotopes according to their mass fragmentation. An experimental study was then carried out in order to evaluate the affinity of the D₅-ethyl-β-D-glucuronide and ¹³C-D₅-ethyl-N-acetylglucosamine for their respective enzyme (β-glucuronidase and N-acetyl glucosaminidase). For this, the efficacy of hydrolysis of each of these enzymes was studied in the presence of their respective probe (FIG. 6) then in the presence of the substrate probe of its partner (FIG. 7). In the first case, each enzyme has hydrolyzed its own probe, thus generating a labeled ethanol signal (FIG. 6). In the other case, no ethanol signal was detected, demonstrating that β-glucuronidase does not hydrolysis the ¹³C-D₅-ethyl-N-acetylglucosamine, and that conversely, the N-acetyl glucosaminidase does not hydrolyze the D₅-ethyl-β-D-glucuronide (FIG. 7). In the presence of the two substrates, each enzyme gives a signal only for the probe specific to it (FIG. 8). This study demonstrates the specificity of the two enzymes for their respective probes.

Two additional probes, D₂-ethyl-β-D-galactopyranoside (probe (3)) and D₄-ethyl-1-α-L-fucopyranoside (probe (4)) respectively targeting β-D-galactosidase and α-L-fucosidase were studied, thus taking the number of probes in the cocktail to 4. In order to differentiate the four isotopes from the ethanol released after hydrolysis of these 4 probes, the MRM transitions of each isotope were re-optimized (Table 3). The absence of interacting responses between the 4 isotopes was then verified by measuring the signal intensity of each isotope, when studied alone or as a mixture, doing so in equimolar concentrations.

TABLE 3
MRM transitions of the 4 isotopes of ethanol. The transitions
indicated in bold were used to quantify each VOC.

| | | Pre- | Frag- | Collision | Residence |
|---|---|---|---|---|---|
| | | cursor | ment | energy | time |
| | | ion | ion | (eV) | (sec) |
| Ethanol-D₂ | 48.1 > 33.1 | 48.1 | 33.1 | 5 | 0.00547 |
| | 48.1 > 46.1 | 48.1 | 46.1 | 5 | 0.00547 |
| | 46.1 > 29.1 | 46.1 | 29.1 | 10 | 0.00547 |
| Ethanol-D₄ | 50.1 > 32.1 | 50.1 | 32.1 | 10 | 0.00547 |
| | 48.1 > 30.1 | 48.1 | 30.1 | 10 | 0.00547 |
| | 49.1 > 45.1 | 49.1 | 45.1 | 15 | 0.00547 |
| Ethanol-Dₛ | 51.1 > 33.1 | 51.1 | 33.1 | 10 | 0.00547 |
| | 51.1 > 30.1 | 51.1 | 30.1 | 10 | 0.00547 |
| Ethanol-¹³CD₅ | 52.1 > 34.1 | 52.1 | 34.1 | 6 | 0.00547 |
| | 52.1 > 50.1 | 52.1 | 50.1 | 4 | 0.00547 |

The cocktail, thus consisting of 4 probes (D₅-ethyl-β-D-glucuronide, ¹³C-D₅-ethyl-N-acetylglucosamine, D₂-ethyl-β-D-galactopyranoside and D-ethyl-1-α-L-fucopyranoside), targets enzymes identified in different types of solid tumors or in the blood circulation of cancer patients.

In order to verify the diagnostic efficacy of this cocktail, it was tested on samples of plasma taken from 4 patients suffering from breast cancer and 3 patients with lung cancer. These patients were diagnosed, but had at the time of sampling not yet received treatment suited to their pathological condition.

After adding the 4 probes (according to the above protocol), the quantity of the 4 isotopes of the ethanol released above the samples of the patients was measured over time. The responses of each isotope were compared with those obtained when the cocktail was added to plasmas of 4 healthy patients. An experimental blank was also carried out each day of analysis. These blanks correspond to negative controls wherein the cocktail was added, whereas none of the 4 enzymes was present therein (FIG. 9). Three probes (D₅-ethyl-β-D-glucuronide, D-ethyl-1-α-L-fucopyranoside, ¹³C-D₅-ethyl-N-acetylglucosamine) allow clear discernment between healthy patients and cancer patients. This result therefore suggests that the 3 enzymes targeted here are secreted at the bronchopulmonary and mammary tumors. They are thus found in the patient's blood circulation, and have maintained some activity (suggesting that they were not denatured or were not very denatured).

On the other hand, the probe D₂-ethyl-β-D-galactopyranoside does not make it possible to discern healthy patients from cancer patients. The enzyme targeted by this probe, β-D-galactosidase, does not appear to be active in the plasma of diseased patients. The underlying assumptions would be that the enzyme is not present at the tumor micro-environment (no secretion by tumor cells or immune cells), or if it is present, that it was made inactive after denaturation.

Finally, these same plasma samples were tested with the reference method targeting 3-D-glucuronidase (UV detection in the presence of para-nitrophenol; protocol described page 5). According to this protocol, the enzyme was not detected in any of the samples, contrary to the protocol using the glucuronylated probe. Such an absence of signal demonstrates the sensitivity of the probes based on VOCs compared to the current standards.

This cocktail of 4 probes was then tested in vivo in order to demonstrate (1) its efficacy for differentiating healthy animals from diseased animals, and the presence of one or more of the target enzymes in the microenvironment of the tumors, in real time.

In the context of this study, the quantity of 4 expired isotopes by mouse models carrying a tumor xenograft (tumor cells of the nasopharynx KB; n=6) was compared to that exhaled by healthy animals (n=6) after i.v. injection of the cocktail.

The concentration of the 4 probes to be injected was determined beforehand according to a precise experimental plan having the objective of determining the amount of each probe inducing a minimum ethanol response in healthy animals (signal reaching a low or even zero basal level). In this context, the healthy animals received increasing doses of the 4 probes (0.5 μg·kg·kg ⁻¹ at 100 μg·kg⁻¹). The animals were then placed in a hermetic cage for 1 h. The amount of each ethanol isotope present in the breath of the animals was then measured for 30 min via an SPME sample, then a GC-TQMS analysis.

The optimal injection doses proved to be different for each probe (that is 1 μg·kg⁻¹ of D₂-ethyl-β-D-galactopyranoside, 10 μg·kg⁻¹ of D₅-ethyl-β-D-glucuronide and ¹³C-D₅-ethyl-N-acetylglucosamine, and finally 100 μg·kg⁻¹ of D-ethyl-1-α-L-fucopyranoside). This result is representative of the variability of penetration/metabolism of each probe, or of the variation of activity of the 4 targeted enzymes.

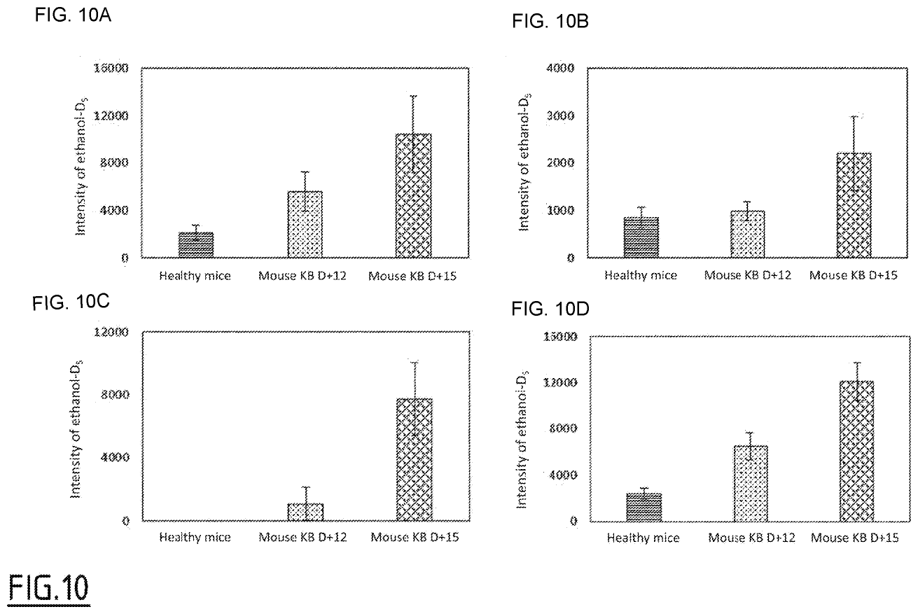

Once the i.v. doses are set, the cocktail was injected into healthy mice and diseased mice after 12 and then 15 days of implantation of the xenograft. The amount of the ethanol isotopes exhaled by the animals was measured 1 h after injection of the cocktail (FIG. 10). A negative control on two mice (healthy and diseased) was carried out. This control consisted of measuring the quantity of 4 isotopes of ethanol in the air surrounding the mice which had not received the cocktail. Ethanol-D₅, ethanol-D4 and ethanol-¹³CD₅ were not detected. On the other hand, the specific transitions of ethanol-D2 gave an important signal, which implies the presence of an interfering compound in the environment of the mice. The amount of ethanol-D2 exhaled by the healthy mice was therefore subtracted from this background noise.

Remarkably, diseased animals were able to be discerned from healthy animals from 12 days after implanting the tumors (minimum duration to have tumor implantation in animals) with this cocktail (the expired amount of 3 isotopes out of 4 was significantly greater for diseased animals).

Three days later, whereas the volume of the tumors had further increased, the 4 probes were activated significantly in the microenvironment of the KB tumors, leading to a significant release of the 4 isotopes of ethanol into the air exhaled by the animals. Thus, diseased animals were able to be unambiguously discerned from healthy animals.

It should be noted that while the D2-ethyl-β-d-galactopyranoside probe does not make it possible to differentiate healthy patients from breast cancer or lung cancer patients, it is activated here in the microenvironment of the nasopharyngeal tumors. Thus, this probe makes it possible to discern the tumors by their location.

### Example 4: Limit of Detection of Three Target Enzymes

The limit of detection (LOD) of a model β-glucuronidase (β-glucuronidase of E. coli; liquid solution 50% glycerol, 140 U·mL⁻¹; Sigma Aldrich) was measured in a plasma matrix in the presence of probe D5-ethyl-β-D-glucuronide added to a concentration of 5·10⁵ M. The results were presented in the initial filing.

The limit of detection was determined for three additional enzymes: α-mannosidase, β-galactosidase, N-acetyl glucosaminidase. Thus, calibration straight lines of the “VOC signal=f(U·L⁻¹ activity)” type are carried out for each of these enzymes.

For this, commercial enzymes were used (α-mannosidase of Canavalia ensiformis in liquid solution of ammonium sulfate, 59.5 U·mL⁻¹ Sigma Aldrich; β-galactosidase of Aspergillus oryzae, solid, 13.4 U·mg⁻¹, Sigma Aldrich; bovine kidney N-acetylglucosaminidase in liquid solution of ammonium sulfate, 34.9 U·mL⁻¹ Sigma Aldrich; Xanthomon α-L-fucosidase in liquid solution of TRIS-HCl, 0.1 U·mL⁻¹), and their respective activities measured by means of the D₅-ethyl-α-mannopyranoside probes at a concentration of 10⁻³ M in the test specimen, D2-ethyl-β-d-galactopyranoside at a concentration of 10⁻⁴ M in the test specimen, ¹³C-D₅-ethyl-N-acetylglucosamide at a concentration of 10⁻³ M in the test specimen, and D₄-ethyl-1-α-L-fucopyranoside at a concentration of 10⁻³ M in the test specimen.

In order to establish the calibration straight lines of each of these three enzymes, solutions of increasing concentrations of enzymes were prepared in acetate buffer (pH 5) and then inserted into a 2 mL vial with an insert (final volume of 100 μL in the vial). A volume of 10 μL of probe was added to obtain said concentration of D₅-ethyl-α-mannopyranoside, D₂-ethyl-β-d-galactopyranoside, ¹³C-D₅-ethyl-N-acetylglucosamide or D-ethyl-1-α-L-fucopyranoside in the test specimen.

After hermetic closure and agitation to the vortex of the vial, an SPME (SPME in semi-polar phase of the Car/PDMS, 1 cm long and 75 μm thick type with a 24 ga needle; Supelco) of the gas phase above the sample of a duration of 30 min will be carried out after 2 h, 4 h and 7 h of reaction. Each kinetic point required a vial reaction, and will be carried out in duplicate. The VOCs trapped on the SPME fiber are then analyzed by GC-TQMS. GC-TMS detection of the ethanol isotopes (thus including ethanol-D₅, ethanol-D2, ethanol-¹³CD₅, and ethanol-D4) is carried out with gas chromatography coupled to a triple quadrupole equipped with an E1 source (TSQ 9000, Thermo Fisher Scientific). The system is controlled by the software TSQ 9000 and the results were analyzed with the Xcalibur software (Thermo Fisher Scientific). The analysis conditions are summarized in tables 1 and 2.

TABLE 4
Settings GC and TQ of the method enabling
the analysis of the VOCs.

| | GC-MS Settings | |
|---|---|---|
| GC | Injector temperature (° C.) | 260 |
| Settings | Septum purge and gas saver (mL · min⁻¹) | 5-20 |
| | Flow rate of the gas - helium (mL · min⁻¹) | 1.4 |
| | Split/Splitless | Splitless |
| | GC Program | 0-7 min: 90° C. |
| TQ | Mode | SRM |
| Settings | Ionization energy (eV) | 70 |
| | Scan speed (scans · s⁻¹) | 20 |
| | Temperature of the transfer | 260-260 |
| | line and source (° C.) | |

TABLE 5
MRM transitions of the 4 isotopes of ethanol. The transitions
indicated in bold were used to quantify each VOC.

| | | Pre- | Frag- | Collision | Residence |
|---|---|---|---|---|---|
| | | cursor | ment | energy | time |
| | | ion | ion | (eV) | (sec) |
| Ethanol-D2 | 48.1 > 33.1 | 48.1 | 33.1 | 5 | 0.00547 |
| | 48.1 > 46.1 | 48.1 | 46.1 | 5 | 0.00547 |
| | 46.1 > 29.1 | 46.1 | 29.1 | 10 | 0.00547 |
| Ethanol-D4 | 50.1 > 32.1 | 50.1 | 32.1 | 10 | 0.00547 |
| | 48.1 > 30.1 | 48.1 | 30.1 | 10 | 0.00547 |
| | 49.1 > 45.1 | 49.1 | 45.1 | 15 | 0.00547 |
| Ethanol-Ds | 51.1 > 33.1 | 51.1 | 33.1 | 10 | 0.00547 |
| | 51.1 > 30.1 | 51.1 | 30.1 | 10 | 0.00547 |
| Ethanol-¹³CD₅ | 52.1 > 34.1 | 52.1 | 34.1 | 6 | 0.00547 |
| | 52.1 > 50.1 | 52.1 | 50.1 | 4 | 0.00547 |

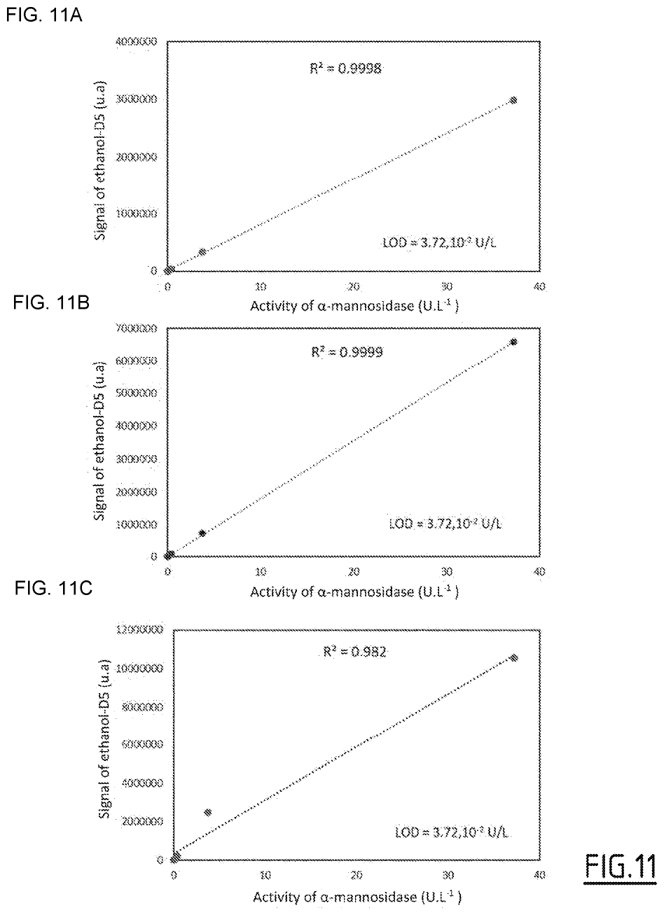

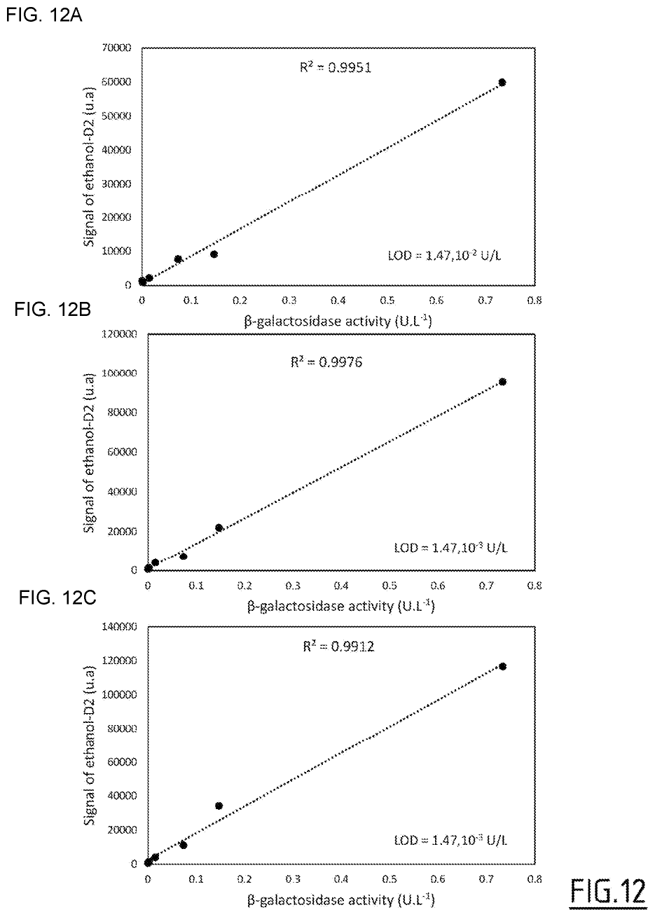

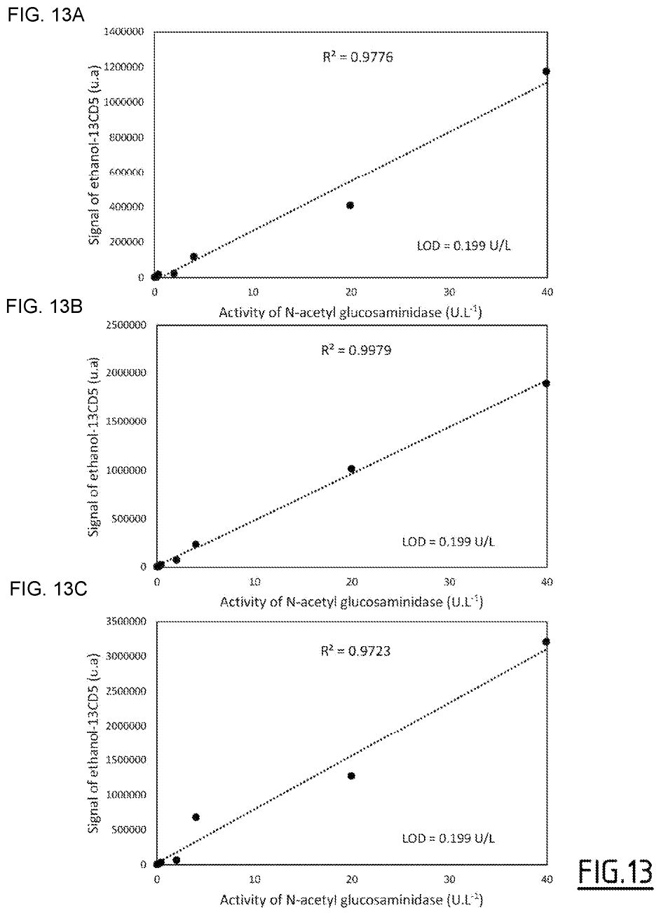

The calibration straight lines of α-mannosidase, β-galactosidase and N-acetyl-glucosaminidase after 2, 4 and 7 h of reaction (30 min of sampling) are presented in FIGS. 11, 12, 13 and 18.

The limit of detection of each of the enzymes is between 1,47·10⁻³ and 0.345 U·L⁻¹ which brings us to a minimum quantity of detectable enzyme less than or equal to 10¹³ mol for the commercial enzymes herein used.

These LODs are 200 to 100,000 times lower than the threshold values obtained with fluorescent probes (DOI: 10.1021/acs.analchem.9b04806; 10.1021/acs.analchem.9b03639; 10.1039/c9tb00175a; 10.1021/acscentsci.0c01189; 10.1039/C8CC10311 A).

### Example 5: Study of the Enzymatic Activities on Cancer Biopsies

A first cocktail consisting of 4 probes (D₅-ethyl-β-D-glucuronide, ¹³C-D₅-ethyl-N-acetylglucosamine, D₂-ethyl-β-D-galactopyranoside and D₄-ethyl-1-α-L-fucopyranoside) was used to demonstrate in vivo its efficacy for differentiating healthy animals from diseased animals, and the presence of one or more of the target enzymes in the tumor microenvironment, doing so in real time. The results obtained demonstrated that the 4 probes were activated significantly in the microenvironment of KB tumors (human cervical tumor), leading to a significant release of the 4 isotopes of ethanol in the air exhaled by the animals.

Thus, diseased animals were able to be unambiguously discerned from healthy animals, only days after implantation of the tumor graft (see results in the examples above).

Anti-cancer agents targeting these enzymatic activities could be of real benefit in treating this type of cancer.

According to this hypothesis, another solid tumor phenotype (tumor MDA-MB-231: human breast cancer cells) was studied with this same probe cocktail, the objective being to identify significant enzymatic activities in order to synthesize the corresponding anticancer agents, targeting these same activities, and finally to verify the therapeutic efficacy thereof.

Since in vivo tests, as mentioned above, are difficult to put in place, we carried out an ex-vivo study directly on tissue.

Thus, and according to the protocol on biopsies described above, tumoral and healthy biopsies (kidneys) were placed in the presence of the cocktail consisting of the 4 probes D₅-ethyl-β-D-glucuronide, ¹³C-D₅-ethyl-N-acetylglucosamine, D₂-ethyl-β-D-galactopyranoside and D-ethyl-1-α-L-fucopyranoside in order to demonstrate the extracellular activity of the corresponding 4 enzymes, respectively targeting β-glucuronidase, the N-acetyl glucosaminidase, β-D-galactosidase and α-1-fucosidase.

Beforehand, the concentration of each probe to be added to the tissues was optimized in order to detect only the enzymes present in the extracellular medium (note: These 4 glycosidases are also present inside the lysosomes of the cells, whether they are cancerous or healthy). Furthermore, and by measurement, the absence of isotopes of ethanol was verified over all the tissues tested.

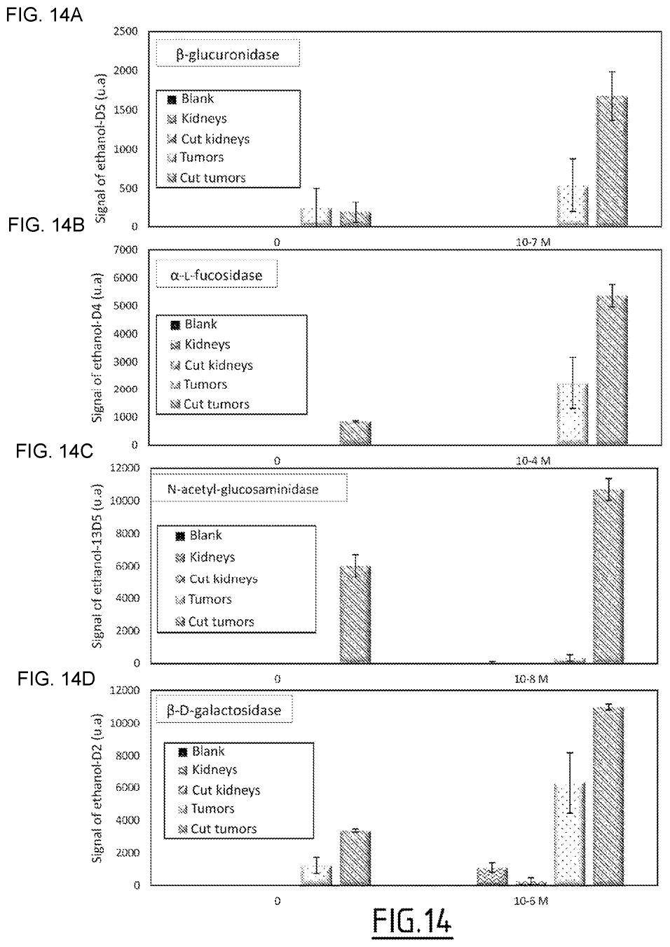

Thus, and according to the protocol described above, the healthy biopsies (healthy whole organs) were placed in the presence of increasing concentrations of each probe. For this, the biopsies were rinsed twice with 500 μL of acetate buffer (pH 5) and then placed in a vial with an insert of 2 μL. A volume of 270 μL of acetate buffer was added to each vial, then the cocktail added so as to obtain a concentration of each of the probes ranging from 10⁴ to 10⁹ M. A concentration was selected for each probe (FIG. 14). It therefore corresponds to the concentration for which the corresponding ethanol isotope signal is small or even absent in healthy tissues (whole or cut; FIG. 14).

The cancerous tissues (whole or cut) were then tested under these conditions.

The results demonstrate that the aforementioned 4 enzymes were detected in the environment of the mammary tumors (FIG. 14). On the other hand, N-acetyl glucosaminidase appears to have an activity greater than the others in the microenvironment of the tumors. An agent targeting this enzyme was then synthesized and then tested in vivo on a mouse model. The results obtained demonstrated a remarkable efficacy of this anticancer agent.

### Example 6: Cocktail for Diagnosis and Prognosis of Diseases: Study of SARS-COV-2 Infection

According to the preliminary results presented in the initial filing, the probes based on VOCs could make it possible to diagnose, predict and monitor the evolution of pathological conditions such as, for example, inflammations (such as cancer) and infections (such as SARS-COV-2 viral infection). In order to exemplify the initial application, an ex vivo study aiming to demonstrate the relevance of the VOCs probes as prognosis tracers of SARS-COV-2 infection was conducted. For this, blood samples from 30 patients infected with SARS-COV-2 were tested using six probes based on VOCs each targeting a different glycololytic activity.

In advance, the enzymatic activities present in the blood samples of 7 healthy volunteers aged 20 to 30 years were studied by means of two cocktails of distinct probes (Table 6). This study had the objective (1) to set the final composition of the cocktails, (2) to define the concentrations of the probes to add to blood samples to obtain a significant isotope response knowing that this response would not be linked to spontaneous hydrolysis of the corresponding probes, (3) to define the optimal kinetic point, (4) to have an idea of the range of response of the isotopes of ethanol in healthy plasma samples.

Furthermore, two samples per patient were carried out in order to study the potential impact on the enzymatic activities here targeted of holding the samples at room temperature for several hours (an action which can take place in hospital conditions). Thus, the first sample was transformed into plasma and then frozen in the hour following the blood sampling. The second was reserved for 5 h at 4° C., before treatment and then freezing. The samples were all stored at −20° C. until analysis.

TABLE 6
Composition of the two cocktails of VOC-based probes

| | Cocktail 1 | | |
|---|---|---|---|
| Enzyme | | α-L- | β- |
| target | β-Galactosidase | Fucosidase | Glucuronidase |
| Probe structure | 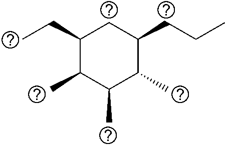 | 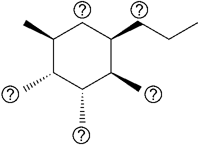 | 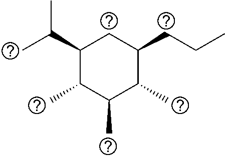 |
| Isotope | Ethanol-D2 | Ethanol-D4 | Ethanol-D5 |
| released | | | |
| Concen- | 10⁻⁴ | 10⁻⁴ | 5 · 10⁻⁵ |
| trations | | | |
| tested | | | |
| (mol · L⁻¹) | | | |
| Injection | Splitless | | |
| mode | | | |
| | Cocktail 1 | Cocktail 2 | |
| Enzyme | N-Acetyl- | | |
| target | glucosaminidase | α-Glucosidase [symbol] | α-Mannosidase |
| Probe structure | 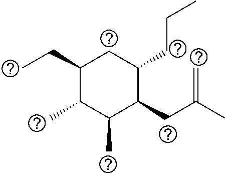 [SMILES: C=C(C)C[C@@H]1[C@@H](CCC)C[C@H](CC)[C@@H](C)[C@@H]1C] | 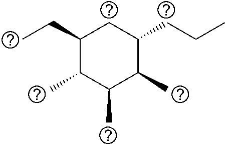 | 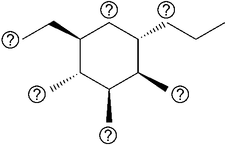 |
| Isotope | Ethanol-13CD5 | Ethanol-D4 | Ethanol-D5 |
| released | | | |
| Concen- | 10⁻⁵ | 10⁻⁴ | 10⁻⁴ |
| trations | | | |
| tested | | | |
| (mol · L⁻¹) | | | |
| Injection | Splitless | Splitless | |
| mode | | | |

[symbol]

indicates data missing or illegible when filed

According to the plasma protocol described above, the probes were used to detect the six enzymatic activities in these samples. To summarize, an aliquot of 25 μL of plasma will be sampled and inserted into a 2 mL vial with an insert. This biological sample was added from 61.4 μL buffer solution (pH 5), 3.6 μL of protease inhibitor (1×) and 10 μL of one or the other of the cocktails. After hermetic closure and agitation to the vortex of the vial, an SPME (SPME in semi-polar phase of the Car/PDMS, 1 cm long and 75 μm thick type with a 24 ga needle, Supelco) of the gas phase above the sample of a duration of 30 min will be carried out after 2 h, 4 h and 7 h of reaction. Each kinetic point requires a vial reaction, and was carried out in duplicate. The VOCs trapped on the SPME fiber are then analyzed by GC-TQMS according to the parameters defined in Tables 4 and 5. Blank cocktails (cocktails in acetate buffer with no plasma) and plasma blanks (plasmas with an acetate buffer without cocktail) were carried out every day of analysis.

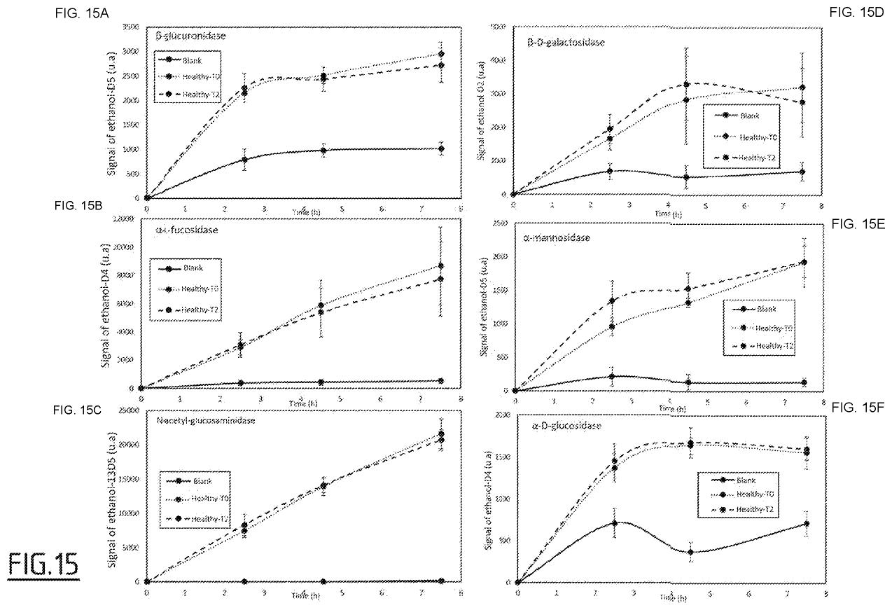

The results obtained (FIG. 15) then demonstrated very distinct activity levels for each of these enzymes. By way of example, the N-acetylglucosaminidase has a very high level of activity compared to β-D-galactosidase, β-glucuronidase, α-mannosidase or α-glucosidase. α-L-fucosidase, meanwhile, has an intermediate activity. Thus, the composition of the cocktails had to be reviewed in order to limit the detection interference between the various isotopes of ethanol. The composition of the cocktails used for the rest of the study is presented in Table 6.

The data obtained also made it possible to demonstrate that 7 h of kinetics were necessary in order to obtain signals for each of the isotopes that emerged from the background noise. Beyond 7 h, an increase in intra-sample variability and spontaneous hydrolysis of the probes were demonstrated, which did not make it possible to maintain the reaction beyond 7 h (tests conducted, data not shown here).

Finally, keeping the blood samples at ambient temperature had no significant impact on the activities targeted here (FIG. 15).

TABLE 7
Composition of the three cocktails of VOC-based probes

| | Cocktail 1 | | |
|---|---|---|---|
| | β- | α- | β- |
| Enzyme target | Galactosidase | Glucosidase | Glucuronidase |
| Probe structure | 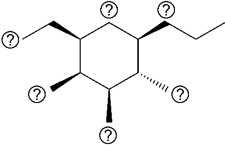 | 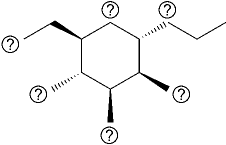 | 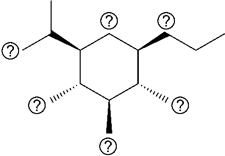 |
| Isotope released | Ethanol-D2 | Ethanol-D4 | Ethanol-D5 |
| Concentrations | 10⁻³/10⁻⁴ | 10⁻³/10⁻⁴ | 5 · 10⁻⁴/5 · 10⁻⁵ |
| tested | | | |
| (mol · L⁻¹) | | | |
| Plasma response | 2000-16000 | 0-3000 | 1000-35000 |
| range (u.a.) | | | |
| Injection mode | Splitless | | |
| | Cocktail 2 | Cocktail 3 | |
| | N-Acetyl- | α-L- | α- |
| Enzyme target | glucosaminidase | Fucosidase [symbol] | Mannosidase |
| Probe structure | 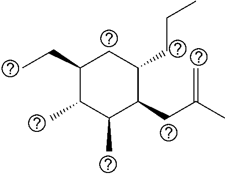 [SMILES: C=C(C)C[C@@H]1[C@@H](CCC)C[C@H](CC)[C@@H](C)[C@@H]1C] | 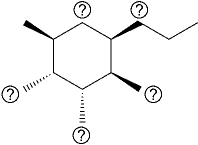 | 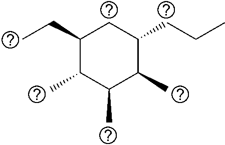 |
| Isotope released | Ethanol-13CD5 | Ethanol-D4 | Ethanol-D5 |
| Concentrations | 10⁻³/10⁻⁴ | 10⁻³/10⁻⁴ | 10⁻³/10⁻⁴ |
| tested | | | |
| (mol · L⁻¹) | | | |
| Plasma response | 10000-300000 | 5000-230000 | 1000-50000 |
| range (u.a.) | | | |

| | | |
|---|---|---|
| Injection mode | Split 1:15 | Splitless |

[symbol]

indicates data missing or illegible when filed

The blood samples of patients infected with SARS-COV-2 were then tested with these three cocktails of probes for 7 h of reaction. A complete experimental plan was made so as to test two concentrations of probes in each sample (concentrations defined in Table 6 above). The objective was here to determine for each of the probes, the concentration making it possible to clearly distinguish between the three groups of patients.

In the context of this study, three groups of patients were studied. The first group consisted of 10 weakly symptomatic patients, aged 39 to 74 years (group A). The second group consisted of 10 hospitalized highly symptomatic patients, aged from 46 to 70 years (group B). The last group consisted of 10 highly symptomatic patients hospitalized in intensive care, aged 50 to 72 years (group C). All these patients had samples taken 8 days after diagnosis.

The responses of the probes in the plasma samples were compared to their responses in so-called “blank” samples and corresponding to the probes added to the acetate buffer alone.

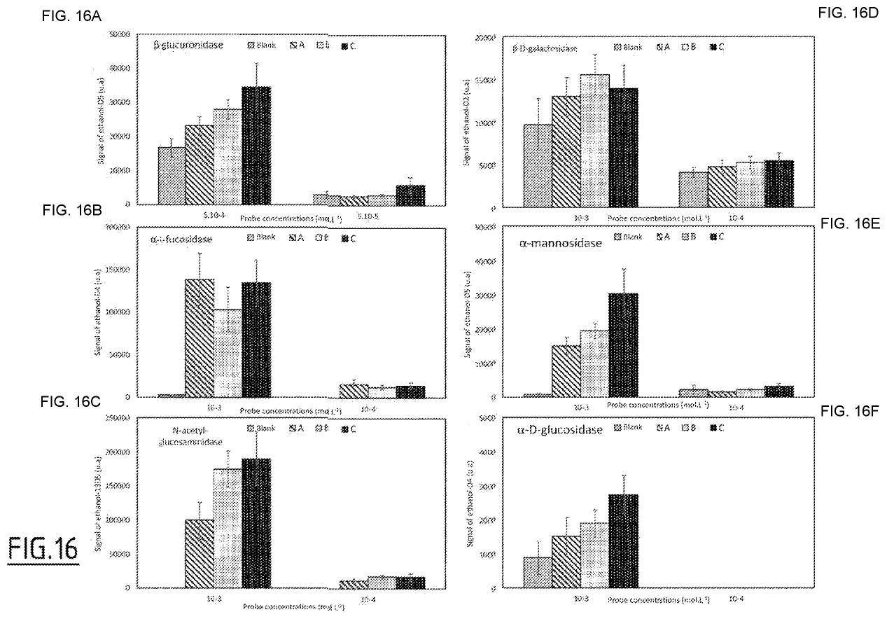

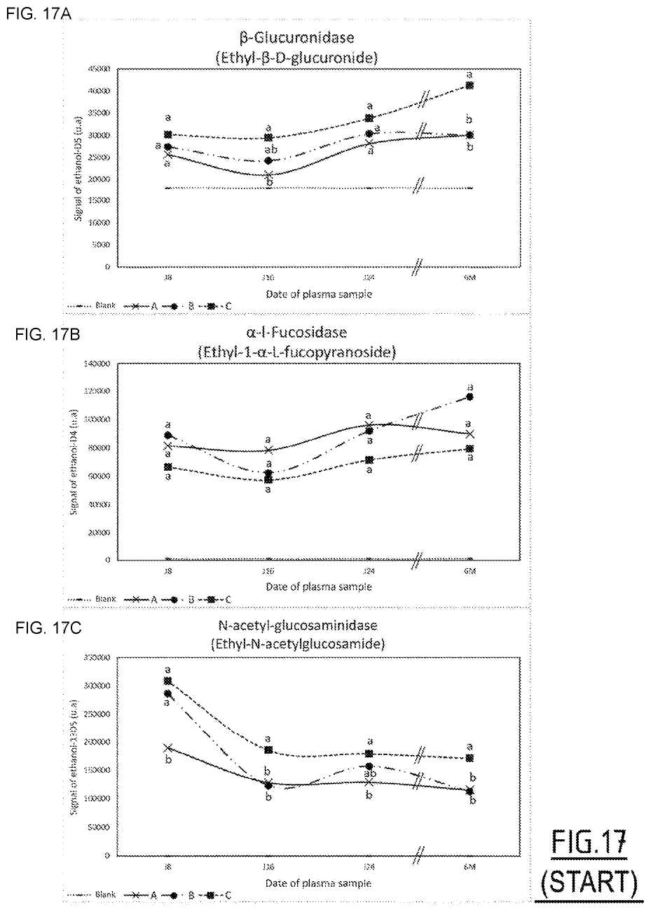

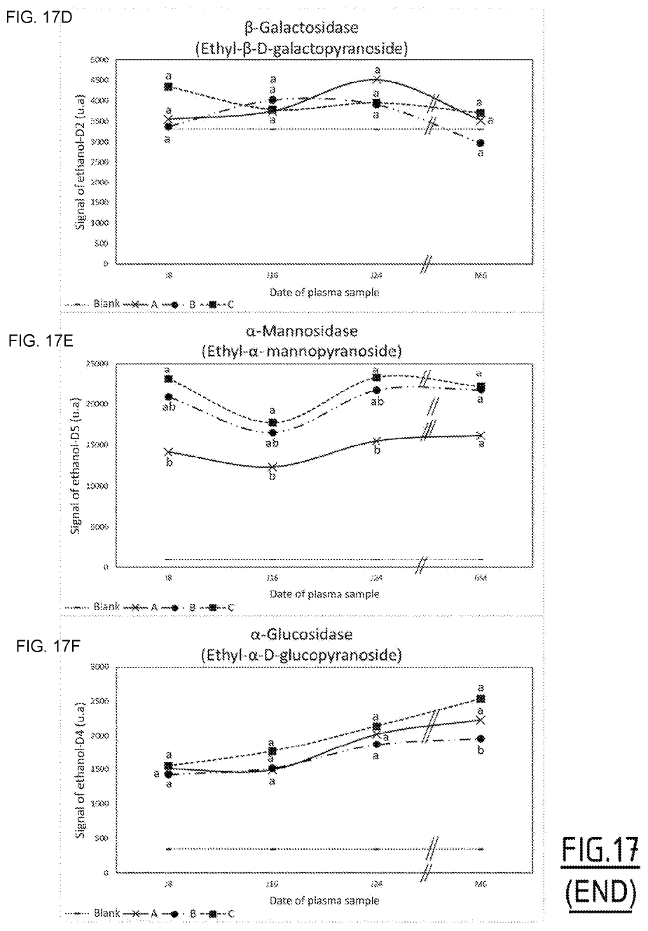

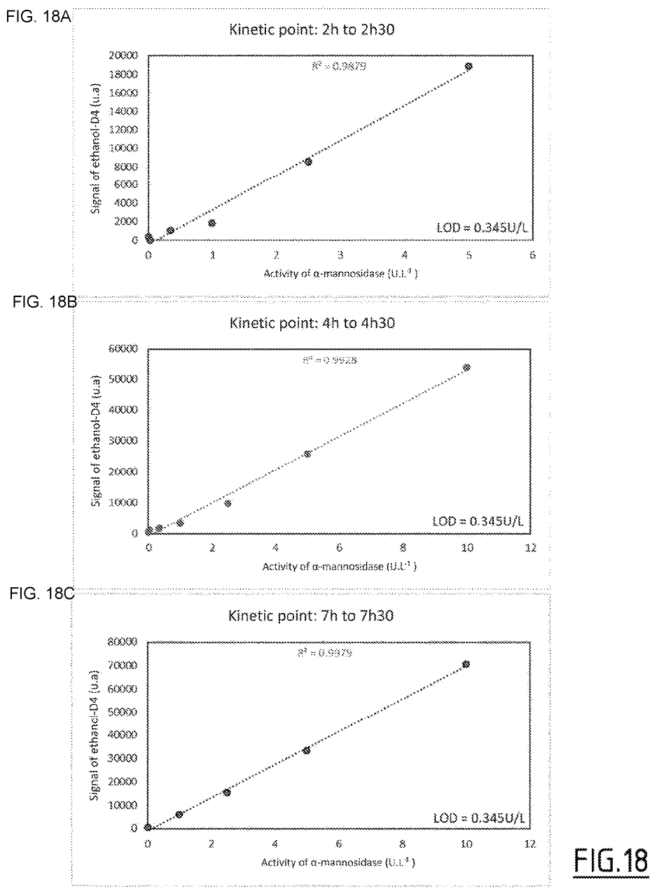

The results demonstrate that the maximum concentrations of the set of probes allow better discrimination between the three groups (included the probe D₅-ethyl-β-D-glucuronide, which nevertheless undergoes significant spontaneous hydrolysis when placing in buffer solution, FIG. 16). The probes will therefore be used now at these concentrations when it will be necessary to test plasma samples.

In addition, the results demonstrate that from 8 days after the positive COVID test, the N-acetylglucosaminidase and α-mannosidase have greater activity in patients who are highly symptomatic and admitted into intensive care. These two enzymes appear to be unfavorable prognostic markers of SARS-COV-2 infection detectable beginning 8 days after infection.

Their concentration significantly reduces the following days, but nevertheless remains high in patients having developed severe symptoms, which makes them SARS-COV-2 infection markers. Furthermore, two other enzymatic markers, β-glucuronidase and α-glucosidase, appear to move into place several weeks after infection in patients admitted into intensive care. Their respective activities become distinguishing 6 months after infection. These two markers would therefore be markers of chronic post-infection inflammation (ARDS) as was developed by patients admitted into intensive care. The tracking of these markers in the longer term would make it possible to verify that the inflammation had receded.

β-D-galactosidase and α-L-fucosidase do not make it possible to distinguish infected patients. However, given that these two enzymes are solid tumor markers, they should allow us to distinguish patients suffering from cancer from those suffering from an infection.

1. A composition comprising:
either at least ethyl-β-D-glucuronide, the ethyl part of which comprises at least one non-radioactive isotope (or ethyl-β-D-glucuronide probe) and at least ethyl-β-D-galactopyranoside (or ethyl-β-D-galactopyranoside probe), the ethyl part of which comprises at least one non-radioactive isotope;
either at least ethyl-α-mannopyranoside, the ethyl part of which comprises at least one non-radioactive isotope (or ethyl-α-mannopyranoside probe); for its use as diagnostic and/or prognostic agent.

2. A use of a composition comprising:
either at least ethyl-β-D-glucuronide, the ethyl part of which comprises at least one non-radioactive isotope (or ethyl-β-D-glucuronide probe) and at least ethyl-β-D-galactopyranoside (or ethyl-β-D-galactopyranoside probe), the ethyl part of which comprises at least one non-radioactive isotope;
either at least ethyl-α-mannopyranoside, the ethyl part of which comprises at least one non-radioactive isotope (or ethyl-α-mannopyranoside); as an agent for the diagnosis and/or prognosis ex vivo of a pathological condition selected from the group consisting of pathological conditions involving an inflammatory reaction in response to a lesion or infection of the organism, chronic inflammatory diseases and acute infections.

3. The use of a composition according to any one of claims 1 or 2, wherein the pathological condition is selected from the group consisting of cancers, bacterial and viral infections, nosocomial infections, autoimmune diseases, respiratory inflammations, chronic inflammations, neurodegenerative diseases, and chronic age-related disorders.

4. A method for diagnosing a pathological condition in a subject, said pathological condition being selected from the group consisting of pathological conditions involving an inflammatory reaction in response to a lesion or infection of the organism, chronic inflammatory diseases, and acute infections,
said method comprising the following steps:
a) adding, in a biological sample of said subject, a composition comprising:
either at least ethyl-β-D-glucuronide, the ethyl part of which comprises at least one non-radioactive isotope (or ethyl-β-D-glucuronide probe) and at least ethyl-β-D-galactopyranoside (or ethyl-β-D-galactopyranoside probe), the ethyl part of which comprises at least one non-radioactive isotope;
or at least ethyl-α-mannopyranoside, the ethyl part of which comprises at least one non-radioactive isotope (or ethyl-α-mannopyranoside probe);
b) measuring the amount of labeled ethanol, released in the gas phase,
c) comparing the values obtained in step b) with a corresponding standard control value, and
d) deducing therefrom whether the subject is affected by said pathological condition.

5. A method for tracking the efficacy of a curative or preventive treatment of a pathological condition in a subject, said pathological condition being selected from the group consisting of pathological conditions involving an inflammatory reaction in response to a lesion or infection of the organism, chronic inflammatory diseases, and acute infections,
said method comprising the following steps:
a) adding a composition comprising:
either at least ethyl-β-D-glucuronide, the ethyl part of which comprises at least one non-radioactive isotope (or ethyl-β-D-glucuronide probe) and at least ethyl-β-D-galactopyranoside (or ethyl-β-D-galactopyranoside probe), the ethyl part of which comprises at least one non-radioactive isotope;
or at least ethyl-α-mannopyranoside, the ethyl part of which comprises at least one non-radioactive isotope (or ethyl-α-mannopyranoside probe);
in a biological sample of said subject at an instant t during said treatment,
b) measuring the amount of labeled ethanol, released in the gas phase,
c) comparing the values obtained in step b) with a corresponding standard control value and/or with a corresponding value obtained before the start of the treatment or at an instant t′ during the treatment which is prior to the instant t,
d) deducing therefrom whether the treatment is effective, and
e) optionally, repeating steps a) to d).

6. A method for tracking the course of a pathological condition in a subject, said pathological condition being selected from the group consisting of pathological conditions involving an inflammatory reaction in response to a lesion or infection of the organism, chronic inflammatory diseases, and acute infections,
said method comprising the following steps:
a) adding, into a biological sample of said subject, at an instant t1, a composition comprising:
either at least ethyl-β-D-glucuronide, the ethyl part of which comprises at least one non-radioactive isotope (or ethyl-β-D-glucuronide probe) and at least ethyl-β-D-galactopyranoside (or ethyl-β-D-galactopyranoside probe), the ethyl part of which comprises at least one non-radioactive isotope;
or at least ethyl-α-mannopyranoside, the ethyl part of which comprises at least one non-radioactive isotope (or ethyl-α-mannopyranoside probe);
b) measuring the amount of labeled ethanol, released in the gas phase, for the sample at instant t1,
c) adding, in a biological sample of said subject, at an instant t2 separated in time from instant t1, the composition as defined in step a),
d) measuring the amount of labeled ethanol, released in the gas phase, for the sample at instant t2,
e) comparing the values obtained in steps b) and d) at instants t1 and t2 respectively,
f) deducing therefrom whether the pathological condition changes favorably, and
g) optionally, repeating steps a) to f).

7. A method for categorizing a subject suffering from a pathological condition in a category C-1 of subjects suffering from said pathological condition at a degree of severity or in a category C-2 of subjects suffering from said pathological condition at another degree of severity, said pathological condition being selected from the group consisting of pathological conditions involving an inflammatory reaction in response to a lesion or infection of the organism, chronic inflammatory diseases and acute infections,
said method comprising the following steps:
a) adding, in a biological sample of said subject, a composition comprising:
either at least ethyl-β-D-glucuronide, the ethyl part of which comprises at least one non-radioactive isotope (or ethyl-β-D-glucuronide probe) and at least ethyl-β-D-galactopyranoside (or ethyl-β-D-galactopyranoside probe), the ethyl part of which comprises at least one non-radioactive isotope;
or at least ethyl-α-mannopyranoside, the ethyl part of which comprises at least one non-radioactive isotope (or ethyl-α-mannopyranoside probe);
b) measuring the amount of labeled ethanol, released in the gas phase,
c) comparing the values obtained in step b) with a corresponding standard control value, and
d) deducing therefrom whether the subject is in the category C1 or in the category C2.

8. The method according to any one of claims 4 to 7, wherein the biological sample is selected from the group consisting of plasma, blood, tissue, saliva, urine, cerebrospinal fluid samples, and biopsies.

9. A kit for the diagnosis of a pathological condition selected from the group consisting of pathological conditions involving an inflammatory reaction in response to a lesion or infection of the organism, chronic inflammatory diseases and acute infections,
said kit comprising a composition comprising:
either at least ethyl-β-D-glucuronide, the ethyl part of which comprises at least one non-radioactive isotope (or ethyl-β-D-glucuronide probe) and at least ethyl-β-D-galactopyranoside (or ethyl-β-D-galactopyranoside probe), the ethyl part of which comprises at least one non-radioactive isotope;
or at least ethyl-α-mannopyranoside, the ethyl part of which comprises at least one non-radioactive isotope (or ethyl-α-mannopyranoside probe).

10. Use of the kit according to claim 9 for the diagnosis of a pathological condition selected from the group consisting of pathological conditions involving an inflammatory reaction in response to a lesion or infection of the organism, chronic inflammatory diseases and acute infections.
